(19) United States
(12) Patent Application Publication
(10) Pub. No.: US 2024/0011040 A1
(43) Pub. Date: Jan. 11, 2024

(54) SALICYLIC ACID-INDUCIBLE GENE EXPRESSION COMPOSITIONS AND SYSTEMS FOR CELLS

(71) Applicant: UNIVERSITY OF HOUSTON SYSTEM, Houston, TX (US)

(72) Inventors: Navin VARADARAJAN, Houston, TX (US); Fatemeh SADEGHI, Houston, TX (US)

(21) Appl. No.: 18/036,488

(22) PCT Filed: Nov. 24, 2021
(86) PCT No.: PCT/US2021/060685

§ 371 Date: May 11, 2023

Related U.S. Application Data
(60) Provisional application No. 63/117,793, filed on Nov. 24, 2020.
(60) Provisional application No. 63/254,862, filed on Oct. 12, 2021.

(51) Int. Cl. C12N 15/63

(52) U.S. Cl. CPC C12N 15/635

(57) ABSTRACT

The present disclosure is directed to composition and methods that enable salicylic acid-inducible gene expression in host cells.

### PRIORITY CLAIM

This application claims benefit of priority to U.S. Provisional Application Ser. No. 63/117,793, filed Nov. 24, 2020, and U.S. Provisional Application Ser. No. 63/254,862, filed Oct. 12, 2021, the entire contents of each of which are hereby incorporated by reference.

### BACKGROUND

### 1. Field of the Disclosure

The present disclosure relates generally to the fields of molecular biology and gene regulation. More particular, the disclosure relates to inducible gene expression systems for use in human cells.

### 2. Background

Inducible control of nuclear localization is a fundamental mechanism naturally employed by cells to enable post-transcriptional control of cellular function and fate. At the molecular level, the change in protein localization is typically facilitated by a change in protein conformation upon ligand binding that exposes a nuclear localization signal (NLS), enabling transport to the nucleus. Identifying proteins or their subdomains responsible for the nucleocytoplasmic shuttling in a ligand-dependent manner has provided tools for diverse applications: (a) basic biology: conditional knockout to study gene functions in specific tissues by fusion to the Cre recombinase (Feil et al., 1996); (b) synthetic biology: inducible CRISPR/Cas9 switches for transcriptional activation and genome editing (Zhang et al., 2019; Zhao et al., 2018); and (c) cell therapies: inducible apoptosis to eliminate therapeutic cells on demand (Liu et al., 2018). Two major systems have been widely utilized to control the inducible translocation of proteins of interest: (a) the estrogen receptor and its ligand tamoxifen (Feil et al., m 1996; Fuhrmann-Benzakein et al., 2000), and (b) rapamycin-induced dimerization of the FK506 binding protein (FKBP) and the FKBP12-rapamycin-binding (FRB) domain of mammalian target of rapamycin (mTOR) (Liu et al., 2018; Xu et al., 2010). These systems are however not without their drawbacks especially for therapeutic applications; tamoxifen, as an estrogen modulator has an impact on cells throughout the body, and rapamycin (and its analogs, rapalogs), can have a direct impact on the essential mTOR pathway in multiple cell types (Di Ventura & Kuhlman, 2016). Thus, improved approaches to facilitate inducible nuclear translocation in response to small molecules are needed.

### SUMMARY

In accordance with the following disclosure, there is provided a method of providing modulated gene expression in a cell comprising (a) providing an engineered cell comprising
(i) a target gene under the control of a transcription element modulated by a transcription modulating protein; and
(ii) a chimeric molecule comprising the transcription modulating protein or functional domain thereof, a salicylic acid binding domain and optionally a further nuclear localization signal, and
   (b) contacting said cell with salicylic acid, thereby modulating expression of said target gene. The chimeric molecule may be encoded from an extrachromosomal element in said cell. The method may further comprise transferring said extrachromosomal element into said cell, such as by liposome or nanoparticle delivery. The chimeric molecule may be expressed from a chromosomal element in said cell. The transcription modulating protein or functional domain therefore may be herpesvirus herpes simplex VP16, FoxA or MyoD. The transcription modulating protein or functional domain thereof may also comprise (i) CRISPR associated protein 9 (Cas9), a dead Cas9 (dCas9), or Cpf1 and said cell is engineered to constitutively express an sgRNA with specificity for the transcription element or (ii) a transcription activator-like effector (TALE).

The salicylic acid binding domain may be from a different protein than the nuclear localization signal. The salicylic acid binding domain may be from the same protein as the nuclear localization signal. The salicylic acid binding domain may be from non-expressor of pathogenesis related gene (NPR) to a TGA transcription factor. The nuclear localization signal may be from SV40. The cell may be a mammalian cell, such as one located in a living subject. The target gene and the transcription element may be native to said cell or the target gene and the transcription element are not native to said cell. The transcription modulating protein or functional domain thereof may be a negative modulator of transcription or may be an inducer of transcription. The target gene may be a chimeric antigen receptor, an antibody, a toxin, a cytokine, an enzyme, a hormone, or a receptor ligand. The target gene may be insulin, a type I interferon, a type II interferon, a type III interferon, an interleukin, erythropoietin, or tissue plasminogen activator. The transcription modulating protein or functional domain thereof may comprise a solubility/folding domain.

Also provided is a method of inducing apoptosis in a mammalian cell (e.g., an immune cell), such as one located in a living subject, comprising (a) providing a mammalian cell engineered to contain a cytotoxic gene (e.g., a DNase) fused to a salicylic acid binding domain and nuclear localization signal, and (b) contacting said cell with salicylic acid, thereby translocating the cytotoxic gene product into the nucleus, resulting in apoptosis.

In another embodiment, there is provide a method of providing modulated gene expression in a cell, such as one located in in living organism, comprising (a) providing a cell comprising:
(i) a heterologous target gene under the control of a transcription element modulated by a salicylic acid responsive transcription modulating protein; and
(ii) a chimeric molecule comprising a functional domain of the salicylic acid responsive transcription modulating protein, a salicylic acid binding domain, and nuclear localization signal, and
   (b) contacting said cell with salicylic acid, thereby modulating expression of said target gene. The cell may be a mammalian cell or a plant cell. The salicylic acid responsive transcription modulating protein further may comprise a nuclear localization domain and/or a solubility/folding domain and/or a transactivation domain. The nuclear localization domain may be from SV40 and/or the transactivation domain comprises herpes simplex virus VP16, FoxA or MyoD. The salicylic acid responsive transcription modulating protein may be from a plant cell. The salicylic acid responsive transcription modulating protein may be from non-expressor of pathogenesis related gene (NPR) or TGA transcription factor.

The target gene may be non-native to said cell. The salicylic acid responsive transcription modulating protein and transcription element may be non-native to said cell. The salicylic acid responsive transcription modulating protein, transcription element and target gene may be non-native to said cell. The target gene may be a chimeric antigen receptor, an antibody, a toxin, a cytokine, an enzyme, a hormone, or a receptor ligand. The target gene may be insulin, a type I interferon, a type II interferon, a type III interferon, an interleukin, erythropoietin, or tissue plasminogen activator. The cell may be a eukaryotic cell. The salicylic acid responsive transcription modulating protein may be a repressor of transcription or an inducer of transcription.

In yet another embodiment, there is provided a method of providing modulated gene expression in a cell comprising (a) providing a host cell comprising:
(i) a heterologous target gene under the control of a transcription element modulated by a salicylic acid responsive transcription factor; and
(ii) a salicylic acid responsive transcription factor, and
   (b) contacting said cell with salicylic acid, thereby modulating expression of said target gene.

In a further embodiment, there is provided a method of inducible gene editing in a cell comprising (a) providing an engineered cell comprising:
(i) a target gene that needs to be edited; and
(ii) a chimeric molecule comprising a nuclease or functional domain thereof, a salicylic acid binding domain and nuclear localization signal, and
   (b) contacting said cell with salicylic acid, thereby editing the target gene.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-C. Controls for the localization experiment were established in HEK293T cells. (FIG. 1A) Design strategy of the plasmid constructs. (FIG. 1B) Sample images obtained using confocal microscope for the controls, and mCherry. Delta NPR1 with and without SA. (FIG. 1C) Pearson coefficients measured by JaCop plugin on ImageJ.

FIGS. 2A-C. (FIG. 2A) The designed constructs for stable transfection into HEK293 cells. (FIG. 2B) Histogram of the mCherry+ sorted cells. Black lines represent un-transfected negative control and the red lines are the transfected cells. (FIG. 2C) Quantitation results of SA-dependent protein nucleocytoplasmic localization.

FIGS. 3A-B. The kinetics of nuclear translocation of the constructs in HEK293 cells. The cells were imaged before the addition of SA and the increased localization of mCherry was quantified within the nucleus as a function of time after the addition of SA.

FIGS. 4A-K. Fusion to NPR1-TAD facilitates translocation of the mCherry reporter protein. (FIG. 4A) Constructs depicting mCherry and mCherry-NLS under CMV promoter in a pcDNA-based vector. (FIG. 47B) Microscopy images representing intracellular localization of mCherry and mCherry-NLS. HEK 293T cells were stained with DAPI after transient transfection with mCherry and mCherry-NLS. mCherry-NLS protein is significantly localized in the nucleus as opposed to the more diffuse mCherry protein as indicated by (FIG. 4C) PCC and (FIG. 4D) Percentage of N, C, and D distributions. At least 30 single cells from one of three representative experiments are shown. A t-test was used for comparing the two distributions. *** p-value<0.001. (FIG. 4E) Domain architecture of NPR1 illustrates that the NLS is incorporated in the C-terminal TAD domain. (FIG. 4F) The modeled structure of the NPR1 predicts six helix bundles in the full-length protein. (FIG. 4G) The design strategy of pcDNA-based plasmid vector expressing NPR1-TAD under CMV promoter for transient transfection experiments. (FIG. 4H) Workflow showing SA-induced protein translocation after transient expression in HEK 293T mammalian cells. (FIG. 4I) Representative confocal microscopy images of DAPI (nucleus), mCherry, and the merged channels showing an increased diffuse expression of mCherry after SA treatment. (FIG. 4J) Bargraph illustrating the percentage of cells with nuclear (N), cytoplasmic (C), or distributed (D) protein. Treatment of mCherry-NPR1-TAD with SA altered the behavior of this fusion protein from predominantly cytoplasmic to diffuse localization. The error bars represent the SEM of three independent trials. (FIG. 4K) Pearson's coefficients were computed for mCherry-NPR1-TAD in the absence and presence of SA confirming its SA-dependent translocation. The error bars represent the SEM. At least 30 single cells from one of three representative experiments are shown. A t-test was used for comparing the two distributions. p-value<0.0001.

FIGS. 5A-C. Generation of stable mCherry NPR1 fusion proteins in HEK 293T cells. (FIG. 5A) Schematics of the constructs designed for stable expression of mCherry-NPR1-TAD, mCherry-Linker-NPR1, NPR1-TAD-mCherry, and NPR1-Linker-mCherry under EF-1α promoter. (FIG. 5B) Stable expression of NPR1 fusion proteins was obtained by lentiviral transduction of HEK 293T cells. The infected cells were collected and sorted for the cells containing mCherry fluorescent protein. (FIG. 5C) Representative histograms confirming successful stable transfection of the NPR1 proteins (red line) versus the un-infected HEK 293T cells (black line).

FIGS. 6A-I. SA inducible translocation of mCherry NPR1 fusion proteins. Representative microscopy images of DAPI (nucleus), mCherry, and the merged channels in the absence and presence of 2.5 mM SA for (FIG. 6A) mCherry-NPR1-TAD, (FIG. 6B) NPR1-TAD-mCherry, and (FIG. 6C) NPR1-mCherry confirming SA-dependent translocation of the NPR1 constructs. Bar graphs reporting the percentage of cells with N, C, or D localization for (FIG. 6D) mCherry-NPR1-TAD, (FIG. 6E) NPR1-TAD-mCherry, and (FIG. 6F) NPR1-mCherry. PCC values for the overlap of mCherry with the nucleus for (FIG. 6G) mCherry-NPR1-TAD, (FIG. 6H) NPR1-TAD-mCherry, and (FIG. 6I) NPR1-mCherry. The PCC numbers are computed using JaCop plugin on ImageJ. At least 30 single cells from one of three representative experiments are shown. A t-test was used for comparing the two distributions. * p-value<0.05, and ** p-value<0.01. For panels D-I the error bars represent the SEM from three independent experiments.

FIGS. 7A-J. Tracking the dynamics of SA-mediated protein translocation at the single-cell level. (FIG. 7A) Illustration of assay procedure including addition of transfected cells into the nanoliter mesh microarray followed by imaging of individual wells before and after SA treatment. Representative images of DAPI (nucleus), mCherry, and the merged channels of a single cell captured at time 0, 7 h, and 24 h after SA addition for (FIG. 7B) mCherry-NPR1-TAD, (FIG. 7C) NPR1-TAD-mCherry, and (FIG. 7D) NPR1-mCherry. (FIG. 7E) The percentage of fusion proteins with predominantly cytoplasmic expression decreased in mCherry-NPR1-TAD. Predominantly nuclear localization was increased in (FIG. 7F) NPR1-TAD-mCherry and (FIG. 7G) NPR1-mCherry fusion proteins. Time-dependent increase in PCC was observed in (FIG. 7H) mCherry-NPR1-TAD, (FIG. 7I) NPR1-TAD-mCherry, (FIG. 7J) NPR1-mCherry. The PCC was computed for the overlap of mCherry and the nucleus. ANOVA was used for comparing the distributions. * p-value<0.05, ** p-value<0.01, *** p-value<0.001, and **** p-value<0.0001. For panels E-J the error bars represent the SEM from three independent experiments.

FIGS. 8A-J. SA-mediated translocation of NPR1 fusion proteins is reversible. (FIG. 8A) Reversibility was investigated by treatment of the transfected cells with SA for 30 h followed by withdrawal of SA. Cells were imaged 18 h post removal of SA. Single-cell images of DAPI (nucleus), mCherry, and the merged channels at 0 h, 20 h, 30 h, and 48 h (18 h after removal of SA) of (FIG. 8B)mCherry-NPR1-TAD, (FIG. 8C)NPR1-TAD-mCherry, and (FIG. 8D) NPR1-mCherry. Removal of SA at 48 h led to (FIG. 8E) increase in predominantly cytoplasmic expression in mCherry-NPR1-TAD from 30 h to 18 h post removal of SA. Decrease in nuclear localization in (FIG. 8F) NPR1-TAD-mCherry and (FIG. 8G) NPR1-mCherry. The error bars represent the SEM from three independent experiments. Time-dependent increase in PCC was observed in (FIG. 8H) mCherry-NPR1-TAD, (FIG. 8I) NPR1-TAD-mCherry, (FIG. 8J) NPR1-mCherry. The PCC was computed for the overlap of mCherry and the nucleus. At least 30 single cells from one of two representative experiments are shown. ANOVA was used for comparing the distributions. * p-value<0.05, ** p-value<0.01, *** p-value<0.001, and **** p-value<0.0001.

FIGS. S1A-C. Cellular localization of mCherry-Linker-NPR1 did not change in response to SA. (FIG. S1A) Microscopy images indicating that distribution of the fusion protein was largely unaltered after SA addition. Analyses using (FIG. S1B) Percentage of N distributed cells, and (FIG. S1C) PCC confirms this result. A t-test was used for comparison of the two distributions. ns: not significant (p-value>0.05) Each dot represents a single cell, the central line denotes the mean, and the error bars denote SEM.

FIGS. S2A-C. Representative examples of cells with differential localization of mCherry. PCC value of a cell with (FIG. S2A) predominantly cytoplasmic expression (FIG. S2B) predominantly nuclear expression, and (FIG. S2C) diffuse expression.

FIGS. S3A-C. NPR1 mediated translocation is unaffected by time in absence of SA. PCC values at 0 h and 24 h for HEK293 cells stably expressing: (FIG. S3A) mCherry-NPR1-TAD, (FIG. S3B) NPR1-TAD-mCherry, and (FIG. S3C) NPR1-mCherry. Each dot represents a single cell, the central line denotes the mean, and the error bars denote SEM. A t-test was used for comparison of the two distributions. ns: not significant FIGS. S4A-F. The translocation efficiency of NPR1 fusions is dependent on the concentration of SA. Representative images of individual cells stained and imaged for DAPI (nucleus), mCherry, and the merged channels of cells captured at 0 mM, 1 mM, and 2.5 mM for (FIG. S4A) mCherry-NPR1-TAD (FIG. S4C) NPR1-TAD-mCherry, and (FIG. S4E) NPR1-mCherry. Relative change in average PCC values after addition of SA at 1 μM-2.5 mM SA for HEK293 cells stably expressing: (FIG. S4B) mCherry-NPR1-TAD, (FIG. S4D) NPR1-TAD-mCherry, and (FIG. S4F) NPR1-mCherry. Each point on the graph represents the relative change in average PCC values comparing at least 30 cells between the tested condition and no SA. The error bars represent SEM of the difference in average PCC values between tested condition and no SA.

### DESCRIPTION OF ILLUSTRATIVE EMBODIMENTS

Inducible control of nuclear localization is a fundamental mechanism naturally employed by cells to enable post-transcriptional control of cellular function and fate. At the molecular level, the change in protein localization is typically facilitated by a change in protein conformation upon ligand binding that exposes a nuclear localization signal (NLS), enabling transport to the nucleus. Identifying proteins or their subdomains responsible for the nucleocytoplasmic shuttling in a ligand-dependent manner has provided tools for diverse applications: (a) basic biology: conditional knockout to study gene functions in specific tissues by fusion to the Cre recombinase; (b) synthetic biology: inducible CRISPR/Cas9 switches for transcriptional activation and genome editing; and (c) cell therapies: inducible apoptosis to eliminate therapeutic cells on demand.

Salicylic acid (SA), derived from plants, has been used medicinally in humans since antiquity. This extensive history of the safety of human use of SA prompted us to investigate proteins or subdomains that can enable nucleocytoplasmic shuttling in response to SA. Within plants, SA is a hormone that plays a key role in innate immunity via the induction of systemic acquired resistance (SAR). Studies suggest that pathogen infection results in the accumulation of SA in infected tissues and distal leaves of the plant and this precedes the upregulation of pathogenesis-related genes (PR genes) (Malamy el al., 1990; Metraux et al., 1990; Tsuda et al., 2009). In Arabidopsis thaliana, both the nonexpresser of PR genes 1 (NPR1) protein and NPR3/4 proteins function as SA receptors (Fu et al., 2012; Wu et al., 2012). NPR1 functions as a transcriptional co-activator whereas NPR3/4 function as transcriptional co-repressors of the expression of genes associated with plant immunity (Ding et al., 2018). Although the function of NPR1/3/4 as SA receptors is established, the exact nature and outcome of the molecular interaction between NPR1 and SA remains controversial. Many different roles have been proposed for this interaction between SA and NPR1 including influencing the oligomerization state, nuclear translocation, and promoting the interaction between NPR1 and NPR3/4 (Tada et al., 2008; Rochon et al., 2006; Kinkema etal., 2000).

In this study, the inventors utilized the fluorescent protein mCherry as the reporter to investigate the ability of SA to induce nuclear translocation of the full-length NPR1 protein or its C-terminal transactivation (TAD) domain using a heterologous system. Their rationale for using the mammalian expression system, HEK293 cells, was that all the other accessory proteins including NPR3/NPR4 are absent in these cells, and is thus ideally suited for directly studying the impact of SA induced conformation changes in NPR1. The results illustrate that the C-terminal TAD of NPR1 is sufficient to enable the SA mediated nuclear translocation of mCherry. Systematic analyses show that fusion proteins containing either full-length NPR1 or NPR1-TAD are capable of nuclear translocation in response to SA. The response to SA is reversible and the proteins revert to their basal localization upon withdrawal of SA. These studies advance a basic understanding of nuclear translocation mediated by the TAD of NPR1 and provide a biotechnological tool for ligand-induced reversible nuclear localization.

Thus, the disclosure describes the design and characterization of a salicylic acid (SA)-inducible gene expression system. Noteworthy features of this gene expression system include the small size of the repressor/activator (10-16 kDa), use of an FDA-approved inducer (drug), and its demonstrated repression/de-repression activity. These salicylic-based systems offer opportunities to circumvent such limitations owing to its small size, use of a relatively safe inducer molecule (salicylic acid), and demonstrated function in multiple different cell types.

These and other aspects of the disclosure are described in detail below.

### I. Salicylic Acid Inducible Expression

A central aspect of the disclosure is the use of salicylic acid. Salicylic acid is a lipophilic monohydroxybenzoic acid, a type of phenolic acid, and a beta hydroxy acid (BHA). It has the formula C₇H₆O₃. This colorless crystalline organic acid is widely used in organic synthesis and functions as a plant hormone. It is derived from the metabolism of salicin. In addition to serving as an important active metabolite of aspirin (acetylsalicylic acid), which acts in part as a pro-drug to salicylic acid, it is probably best known for its use as a key ingredient in topical anti-acne products. The salts and esters of salicylic acid are known as salicylates. It is on WHO's List of Essential Medicines, the safest and most effective medicines needed in a health system.

### II. Host Cells

The host cells in accordance with the present disclosure may be virtually any cell including fungal, plant, mammalian and human cells. In particular, the cells may be engineered/recombinant cells. The cells may be cell lines, Chinese hamster ovary cells, human embryonic kidney cells (HEK), stem cells, endothelial cells, immune cells (B cells, T cells, NK cells), dendritic cells, muscle cells epithelial cells, cardiac cells, renal cells, spleen cells, or liver cells.

### III. Control Systems

The present disclosure provides control systems that depend on the use of salicylic acid and salicylic acid responsive proteins. One example includes proteins that translocate to the nucleus in the presence of salicylic acid. Another example is the class of salicylic acid responsive transcription factors that induce or repress gene expression. The following are non-limiting examples of such molecules as well as other molecules that can be used in conjunction with salicylic acid responsive molecules.

A. Plant Transcriptional Control Proteins

Salicylic acid is also an essential hormone in plant immunity, most notably in Arabidopsis thaliana The transcriptional coregulator NPR1 directly binds salicylic acid and is named after the npr1 mutants that failed to activate pathogenesis-related gene expression and were, therefore, defective in resistance to virulent bacteria in local and systemic tissues. Mechanistically, it has been reported that binding of salicylic acid causes a conformational change in NPR1 that is accompanied by the release of the C-terminal transactivation domain from the N-terminal autoinhibitory BTB/POZ domain. NPR1 controls plant immunity and is key to salicylic acid-dependent signaling pathways. Interestingly, the NPR3/NPR4 receptors, which also respond to salicylic acid, act in an opposite fashion as NPR1.

Other species that have been shown to contain NPR1-like proteins suitable for use in accordance with the present disclosure include Malus pumila, Malus hupehensis, Oryza sativa, Populus trichocarpa, Nicotiana tabacum, Nicotiana glutinosa, Vitis vinifera, Vitis aestivalis c.v. Norton, Gossypium hirsutum, Pyrus pyrifolia, Ipomoea batatas, Carica papaya, Musa acuminata, Musa spp. ABB, Solanum lycopersicum, Brassica juncea, Glycine mas, Theobroma cacao, Saccharum spp., Coffea arabica, Phalaenopsis aphrodite, Triticum aestivum L., Beta vulgaris. Persea americana, Cocos nucifera L.. Gladiolus hybridus, Brassica napus, Arachis hypogaea, Lilium ‘Sorbonne’ and Eucalyptus grandis. See also Pokotylo et al. (2019), incorporated herein by reference.

B. Additional Proteins/Domains

In addition to the salicylic acid responsive proteins discussed above, the transcriptional modulating protein may contain other elements, which are discussed below.

Casp9/Cpf1. CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. The team demonstrated that they could disable one or both sites while preserving Cas9's ability to locate its target DNA. Jinek el al. (2012) combined tracrRNA and spacer RNA into a “single-guide RNA” molecule that, mixed with Cas9, can find and cut the correct DNA targets and such synthetic guide RNAs are used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas systems containing a 1,300 amino acid protein. See also U.S. Patent Publication 2014/0068797, which is incorporated by reference in its entirety.

In some embodiments, the compositions of the disclosure include a small version of a Cas9 from the bacterium Staphylococcus aureus (UniProt Accession No. J7RUA5). The small version of the Cas9 provides advantages over wildtype or full length Cas9. In some embodiments the Cas9 is a spCas9 (AddGene).

Clustered Regularly Interspaced Short Palindromic Repeats from Prevotella and Francisella 1 or CRISPR/Cpf1 is a DNA-editing technology which shares some similarities with the CRISPR/Cas9 system. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

Cpf1 appears in many bacterial species. The ultimate Cpf1 endonuclease that was developed into a tool for genome editing was taken from one of the first 16 species known to harbor it.

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type III promoter U6. Synthetic gRNAs are slightly over 100 bp at the minimum length and contain a portion which is targets the 20 protospacer nucleotides immediately preceding the PAM sequence NGG; gRNAs do not contain a PAM sequence.

In some embodiments, the gRNA targets a site within a wildtype dystrophin gene. In some embodiments, the gRNA targets a site within a mutant dystrophin gene. In some embodiments, the gRNA targets a dystrophin intron. In some embodiments, the gRNA targets a dystrophin exon. In some embodiments, the gRNA targets a site in a dystrophin exon that is expressed and is present in one or more of the dystrophin isoforms shown in Table 1. In embodiments, the gRNA targets a dystrophin splice site. In some embodiments, the gRNA targets a splice donor site on the dystrophin gene. In embodiments, the gRNA targets a splice acceptor site on the dystrophin gene.

TALE. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence, so when combined with a nuclease, DNA can be cut at specific locations. The restriction enzymes can be introduced into cells, for use in gene editing or for genome editing in situ, a technique known as genome editing with engineered nucleases. Alongside zinc finger nucleases and CRISPR/Cas9, TALEN is a prominent tool in the field of genome editing.

TAL effectors are proteins that are secreted by Xanthomonas bacteria via their type III secretion system when they infect plants. The DNA binding domain contains a repeated highly conserved 33-34 amino acid sequence with divergent 12th and 13th amino acids. These two positions, referred to as the Repeat Variable Diresidue (RVD), are highly variable and show a strong correlation with specific nucleotide recognition. This straightforward relationship between amino acid sequence and DNA recognition has allowed for the engineering of specific DNA-binding domains by selecting a combination of repeat segments containing the appropriate RVDs. Notably, slight changes in the RVD and the incorporation of non-conventional RVD sequences can improve targeting specificity.

Other Factors. In addition to the agent mentioned above, the disclosure also contemplates the use of transcriptional activators, nuclear translocation signals and protein stabilization domains, all of which are well known in the field.

### IV. Target Genes

The target genes can be virtually any proteins of interest. There a large number of proteins useful for manufacturing, food preparation, and medicine. Of particular interest are therapeutic/prophylactic proteins.

Based on pharmacological activity, five groups are recognized: (a) replacing a protein that is deficient or abnormal; (b) augmenting an existing pathway; (c) providing a novel function or activity; (d) interfering with a molecule or organism; and (e) delivering other compounds or proteins, such as a radionuclide, cytotoxic drug, or effector proteins. Therapeutic proteins can also be grouped on their molecular type. This includes antibody-based drugs, Fc fusion proteins, anticoagulants, blood factors, bone morphogenetic proteins, engineered protein scaffolds, enzymes, growth factors, hormones, interferons, interleukins, and thrombolytics. Yet another classification scheme is based on mechanism of action including (a) binding non-covalently to target, (b) affecting covalent bonds, and (c) exerting activity without specific interactions. Most current protein therapeutics are recombinant and relate to therapies for cancers, immune disorders, infections, genetic deficiencies and other diseases. Some particular examples are as follows.

A. Interferons

Interferons (IFNs) are a group of signaling proteins made and released by host cells in response to the presence of several viruses. In a typical scenario, a virus-infected cell will release interferons causing nearby cells to heighten their anti-viral defenses. IFNs belong to the large class of proteins known as cytokines, molecules used for communication between cells to trigger the protective defenses of the immune system that help eradicate pathogens. Interferons are named for their ability to “interfere” with viral replication by protecting cells from virus infections. IFNs also have various other functions: they activate immune cells, such as natural killer cells and macrophages; they increase host defenses by up-regulating antigen presentation by virtue of increasing the expression of major histocompatibility complex (MHC) antigens. Certain symptoms of infections, such as fever, muscle pain and “flu-like symptoms”, are also caused by the production of IFNs and other cytokines. More than twenty distinct IFN genes and proteins have been identified in animals, including humans. They are typically divided among three classes: Type I IFN, Type II IFN, and Type III IFN. IFNs belonging to all three classes are important for fighting viral infections and for the regulation of the immune system. IFNα, IFNβ and IFNγ are examples.

B. Interleukins

Interleukins (ILs) are a group of cytokines (secreted proteins and signal molecules) that were first seen to be expressed by white blood cells (leukocytes). ILs can be divided into four major groups based on distinguishing structural features. However, their amino acid sequence similarity is rather weak (typically 15-25% identity). The human genome encodes more than 50 interleukins and related proteins.

The function of the immune system depends in a large part on interleukins, and rare deficiencies of a number of them have been described, all featuring autoimmune diseases or immune deficiency. The majority of interleukins are synthesized by helper CD4 T lymphocytes, as well as through monocytes, macrophages, and endothelial cells. They promote the development and differentiation of T and B lymphocytes, and hematopoietic cells. Interleukin receptors on astrocytes in the hippocampus are also known to be involved in the development of spatial memories in mice.

Interleukin 1 alpha and interleukin 1 beta (IL1 alpha and IL1 beta) are cytokines that participate in the regulation of immune responses, inflammatory reactions, and hematopoiesis. Two types of IL-1 receptor, each with three extracellular immunoglobulin (Ig)-like domains, limited sequence similarity (28%) and different pharmacological characteristics have been cloned from mouse and human cell lines: these have been termed type I and type II receptors. The receptors both exist in transmembrane (TM) and soluble forms: the soluble IL-1 receptor is thought to be post-translationally derived from cleavage of the extracellular portion of the membrane receptors.

Both IL-1 receptors (CD121a/IL1R1, CD121b/IL1R2) appear to be well conserved in evolution, and map to the same chromosomal location. The receptors can both bind all three forms of IL-1 (IL-1 alpha, IL-1 beta and IL-1 receptor antagonist). The crystal structures of IL1A and IL1B have been solved, showing them to share the same 12-stranded beta-sheet structure as both the heparin binding growth factors and the Kunitz-type soybean trypsin inhibitors. The beta-sheets are arranged in 4 similar lobes around a central axis, 8 strands forming an anti-parallel beta-barrel. Several regions, especially the loop between strands 4 and 5, have been implicated in receptor binding.

Molecular cloning of the Interleukin 1 Beta converting enzyme is generated by the proteolytic cleavage of an inactive precursor molecule. A complementary DNA encoding protease that carries out this cleavage has been cloned. Recombinant expression enables cells to process precursor Interleukin 1 Beta to the mature form of the enzyme.

Interleukin 1 also plays a role in the Central Nervous System. Research indicates that mice with a genetic deletion of the type I IL-1 receptor display markedly impaired hippocampal-dependent memory functioning and Long-term potentiation, although memories that do not depend on the integrity of the hippocampus seem to be spared. However, when mice with this genetic deletion have wild-type neural precursor cells injected into their hippocampus and these cells are allowed to mature into astrocytes containing the interleukin-1 receptors, the mice exhibit normal hippocampal-dependent memory function, and partial restoration of long-term potentiation.

T lymphocytes regulate the growth and differentiation of T cells and certain B cells through the release of secreted protein factors. These factors, which include interleukin 2 (IL2), are secreted by lectin- or antigen-stimulated T cells, and have various physiological effects. IL2 is a lymphokine that induces the proliferation of responsive T cells. In addition, it acts on some B cells, via receptor-specific binding, as a growth factor and antibody production stimulant. The protein is secreted as a single glycosylated polypeptide, and cleavage of a signal sequence is required for its activity. Solution NMR suggests that the structure of IL2 comprises a bundle of 4 helices (termed A-D), flanked by 2 shorter helices and several poorly defined loops. Residues in helix A, and in the loop region between helices A and B, are important for receptor binding. Secondary structure analysis has suggested similarity to IL4 and granulocyte-macrophage colony stimulating factor (GMCSF).

Interleukin 3 (IL3) is a cytokine that regulates hematopoiesis by controlling the production, differentiation and function of granulocytes and macrophages. The protein, which exists in vivo as a monomer, is produced in activated T cells and mast cells, and is activated by the cleavage of an N-terminal signal sequence.

IL3 is produced by T lymphocytes and T-cell lymphomas only after stimulation with antigens, mitogens, or chemical activators such as phorbol esters. However, IL3 is constitutively expressed in the myelomonocytic leukemia cell line WEHI-3B. It is thought that the genetic change of the cell line to constitutive production of IL3 is the key event in development of this leukemia.

Interleukin 4 (IL4) is produced by CD4⁺ T cells specialized in providing help to B cells to proliferate and to undergo class switch recombination and somatic hypermutation. Th2 cells, through production of IL-4, have an important function in B-cell responses that involve class switch recombination to the IgG1 and IgE isotypes.

Interleukin 5 (IL5), also known as eosinophil differentiation factor (EDF), is a lineage-specific cytokine for eosinophilpoiesis. It regulates eosinophil growth and activation, and thus plays an important role in diseases associated with increased levels of eosinophils, including asthma. IL5 has a similar overall fold to other cytokines (e.g., IL2, IL4 and GCSF), but while these exist as monomeric structures, IL5 is a homodimer. The fold contains an anti-parallel 4-alpha-helix bundle with a left handed twist, connected by a 2-stranded anti-parallel beta-sheet. The monomers are held together by 2 interchain disulphide bonds.

Interleukin 6 (IL6), also referred to as B-cell stimulatory factor-2 (BSF-2) and interferon beta-2, is a cytokine involved in a wide variety of biological functions. It plays an essential role in the final differentiation of B cells into immunoglobulin-secreting cells, as well as inducing myeloma/plasmacytoma growth, nerve cell differentiation, and, in hepatocytes, acute-phase reactants.

A number of other cytokines may be grouped with IL6 on the basis of sequence similarity. These include granulocyte colony-stimulating factor (GCSF) and myelomonocytic growth factor (MGF). GCSF acts in hematopoiesis by affecting the production, differentiation, and function of 2 related white cell groups in the blood. MGF also acts in hematopoiesis, stimulating proliferation and colony formation of normal and transformed avian cells of the myeloid lineage.

Cytokines of the IL6/GCSF/MGF family are glycoproteins of about 170 to 180 amino acid residues that contain four conserved cysteine residues involved in two disulphide bonds. They have a compact, globular fold (similar to other interleukins), stabilised by the two disulphide bonds. One half of the structure is dominated by a 4-alpha-helix bundle with a left-handed twist; the helices are anti-parallel, with two overhand connections, which fall into a double-stranded anti-parallel beta-sheet. The fourth alpha-helix is important to the biological activity of the molecule.

Interleukin 7 (IL-7) is a cytokine that serves as a growth factor for early lymphoid cells of both B- and T-cell lineages.

Interleukin 8 is a chemokine produced by macrophages and other cell types such as epithelial cells, airway smooth muscle cells and endothelial cells. Endothelial cells store IL-8 in their storage vesicles, the Weibel-Palade bodies. In humans, the interleukin-8 protein is encoded by the CXCL8 gene. IL-8 is initially produced as a precursor peptide of 99 amino acids which then undergoes cleavage to create several active IL-8 isoforms. In culture, a 72 amino acid peptide is the major form secreted by macrophages.

There are many receptors on the surface membrane capable of binding IL-8; the most frequently studied types are the G protein-coupled serpentine receptors CXCR1 and CXCR2. Expression and affinity for IL-8 differs between the two receptors (CXCR1>CXCR2). Through a chain of biochemical reactions, IL-8 is secreted and is an important mediator of the immune reaction in the innate immune system response.

Interleukin 9 (IL-9) is a cytokine that supports IL-2 independent and IL-4 independent growth of helper T cells. Early studies had indicated that Interleukin 9 and 7 seem to be evolutionary related and Pfam, InterPro and PROSITE entries exist for interleukin 7/interleukin 9 family. However, a recent study has shown that IL-9 is, in fact, much closer to both, IL-2 and IL-15, than to IL-7. Moreover, the study showed irreconcilable structural differences between IL-7 and all the remaining cytokines signalling through the γc receptor (IL-2, IL-4, IL-7, IL-9, IL-15 and IL-21).

Interleukin 10 (IL-10) is a protein that inhibits the synthesis of a number of cytokines, including IFN-gamma, IL-2, IL-3, TNF, and GM-CSF produced by activated macrophages and by helper T cells. In structure, IL-10 is a protein of about 160 amino acids that contains four conserved cysteines involved in disulphide bonds. IL-10 is highly similar to the Human herpesvirus 4 (Epstein-Barr virus) BCRF1 protein, which inhibits the synthesis of gamma-interferon and to Equid herpesvirus 2 (Equine herpesvirus 2) protein E7. It is also similar, but to a lesser degree, with human protein mda-7. a protein that has antiproliferative properties in human melanoma cells. Mda-7 contains only two of the four cysteines of IL-10.

Interleukin 11 (IL-11) is a secreted protein that stimulates megakaryocytopoiesis, initially thought to lead to an increased production of platelets (it has since been shown to be redundant to normal platelet formation), as well as activating osteoclasts, inhibiting epithelial cell proliferation and apoptosis, and inhibiting macrophage mediator production. These functions may be particularly important in mediating the hematopoietic, osseous and mucosal protective effects of interleukin 11.

Interleukin 12 (IL-12) is a disulphide-bonded heterodimer consisting of a 35 kDa alpha subunit and a 40 kDa beta subunit. It is involved in the stimulation and maintenance of Th1 cellular immune responses, including the normal host defence against various intracellular pathogens, such as Leishmania, Toxoplasma, Measles virus, and Human immunodeficiency virus 1 (HIV). IL-12 also has an important role in enhancing the cytotoxic function of NK cells and role in pathological Th1 responses, such as in inflammatory bowel disease and multiple sclerosis. Suppression of IL-12 activity in such diseases may have therapeutic benefit. On the other hand, administration of recombinant IL-12 may have therapeutic benefit in conditions associated with pathological Th2 responses.

Interleukin 13 (IL-13) is a pleiotropic cytokine that may be important in the regulation of the inflammatory and immune responses. It inhibits inflammatory cytokine production and synergises with IL-2 in regulating interferon-gamma synthesis. The sequences of IL-4 and IL-13 are distantly related.

Interleukin 15 (IL-15) is a cytokine that possesses a variety of biological functions, including stimulation and maintenance of cellular immune responses. IL-15 stimulates the proliferation of T lymphocytes, which requires interaction of IL-15 with IL-15R alpha and components of IL-2R, including IL-2R beta and IL-2R gamma (common gamma chain, γc), but not IL-2R alpha.

Interleukin 17 (IL-17) is a potent proinflammatory cytokine produced by activated memory T cells. The IL-17 family is thought to represent a distinct signalling system that appears to have been highly conserved across vertebrate evolution.

C. Chimeric Antigen Receptors

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of cancer-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to enable antigen binding. The transmembrane domain is a typical hydrophobic alpha helix usually derived from the original molecule of the signalling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain-linker-heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH₂CH₃ region of immunoglobulin and portions of CD3. For most scFv based constructs, the IgG1 hinge suffices. However the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed. For example, chimeric CD28 and OX40 can be used with CD3-Zeta to transmit a proliferative/survival signal, or all three can be used together.

“First-generation” CARs typically had the intracellular domain from the CD3 ξ-chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third-generation” CARs combine multiple signaling domains, such as CD3z-CD28-41BB or CD3z-CD28-OX40, to further augment potency.

Adoptive transfer of T cells expressing chimeric antigen receptors is a promising anti-cancer therapeutic as CAR-modified T cells can be engineered to target virtually any tumor associated antigen. There is great potential for this approach to improve patient-specific cancer therapy in a profound way. Following the collection of a patient's T cells, the cells are genetically engineered to express CARs specifically directed towards antigens on the patient's tumor cells, then infused back into the patient. Although adoptive transfer of CAR-modified T-cells is a unique and promising cancer therapeutic, there are significant safety concerns. Clinical trials of this therapy have revealed potential toxic effects of these CARs when healthy tissues express the same target antigens as the tumor cells, leading to outcomes similar to graft-versus-host disease (GVHD). A potential solution to this problem is engineering a suicide gene into the modified T cells. In this way, administration of a prodrug designed to activate the suicide gene during GVHD triggers apoptosis in the suicide gene-activated CAR T cells. This method has been used safely and effectively in hematopoietic stem cell transplantation (HSCT). Adoption of suicide gene therapy to the clinical application of CAR-modified T cell adoptive cell transfer has potential to alleviate GVHD while improving overall anti-tumor efficacy.

D. Other Proteins of Interest

Insulin. Insulin is a peptide hormone produced by beta cells of the pancreatic islets; it is considered to be the main anabolic hormone of the body. It regulates the metabolism of carbohydrates, fats and protein by promoting the absorption of glucose from the blood into liver, fat and skeletal muscle cells. In these tissues the absorbed glucose is converted into either glycogen via glycogenesis or fats (triglycerides) via lipogenesis, or, in the case of the liver, into both. Glucose production and secretion by the liver is strongly inhibited by high concentrations of insulin in the blood. Circulating insulin also affects the synthesis of proteins in a wide variety of tissues. It is therefore an anabolic hormone, promoting the conversion of small molecules in the blood into large molecules inside the cells. Low insulin levels in the blood have the opposite effect by promoting widespread catabolism, especially of reserve body fat.

Beta cells are sensitive to blood sugar levels so that they secrete insulin into the blood in response to high level of glucose; and inhibit secretion of insulin when glucose levels are low. Insulin enhances glucose uptake and metabolism in the cells, thereby reducing blood sugar level. Their neighboring alpha cells, by taking their cues from the beta cells, secrete glucagon into the blood in the opposite manner: increased secretion when blood glucose is low, and decreased secretion when glucose concentrations are high. Glucagon increases blood glucose level by stimulating glycogenolysis and gluconeogenesis in the liver. The secretion of insulin and glucagon into the blood in response to the blood glucose concentration is the primary mechanism of glucose homeostasis.

Decreased or loss of insulin activity results in diabetes mellitus, a condition of high blood sugar level (hyperglycemia). There are two types of the disease. In type 1 diabetes mellitus, the beta cells are destroyed by an autoimmune reaction so that insulin can no longer be synthesized or be secreted into the blood. In type 2 diabetes mellitus, the destruction of beta cells is less pronounced than in type 1 diabetes and is not due to an autoimmune process. Instead, there is an accumulation of amyloid in the pancreatic islets, which likely disrupts their anatomy and physiology. The pathogenesis of type 2 diabetes is not well understood but reduced population of islet beta-cells, reduced secretory function of islet beta-cells that survive, and peripheral tissue insulin resistance are known to be involved. Type 2 diabetes is characterized by increased glucagon secretion which is unaffected by, and unresponsive to the concentration of blood glucose. But insulin is still secreted into the blood in response to the blood glucose. As a result, glucose accumulates in the blood.

The human insulin protein is composed of 51 amino acids, and has a molecular mass of 5808 Da. It is a heterodimer of an A-chain and a B-chain, which are linked together by disulfide bonds. Insulin's structure varies slightly between species of animals. Insulin from animal sources differs somewhat in effectiveness (in carbohydrate metabolism effects) from human insulin because of these variations. Porcine insulin is especially close to the human version and was widely used to treat type 1 diabetics before human insulin could be produced in large quantities by recombinant DNA technologies.

tPA. Tissue plasminogen activator (tPA or PLAT) is a protein involved in the breakdown of blood clots. It is a serine protease found on endothelial cells, the cells that line the blood vessels. As an enzyme, it catalyzes the conversion of plasminogen to plasmin, the major enzyme responsible for clot breakdown. Human tPA has a molecular weight of ˜70 kDa in the single-chain form.

tPA can be manufactured using recombinant biotechnology techniques; tPA produced by such means are referred to as recombinant tissue plasminogen activator (rtPA). Specific rtPAs include alteplase, reteplase, and tenecteplase. They are used in clinical medicine to treat embolic or thrombotic stroke. The use of this protein is contraindicated in hemorrhagic stroke and head trauma. The antidote for tPA in case of toxicity is aminocaproic acid.

tPA is used in some cases of diseases that feature blood clots, such as pulmonary embolism, myocardial infarction, and stroke, in a medical treatment called thrombolysis. The most common use is for ischemic stroke. It can either be administered systemically, in the case of acute myocardial infarction, acute ischemic stroke, and most cases of acute massive pulmonary embolism, or administered through an arterial catheter directly to the site of occlusion in the case of peripheral arterial thrombi and thrombi in the proximal deep veins of the leg.

EPO. Erythropoietin EPO), also known as erythropoetin, hematopoietin, or hemopoietin, is a glycoprotein cytokine secreted mainly by the kidney in response to cellular hypoxia; it stimulates red blood cell production (erythropoiesis) in the bone marrow. Low levels of EPO (around 10 mU/mL) are constantly secreted sufficient to compensate for normal red blood cell turnover. Common causes of cellular hypoxia resulting in elevated levels of EPO (up to 10 000 mU/mL) include any anemia, and hypoxemia due to chronic lung disease.

Erythropoietin is produced by interstitial fibroblasts in the kidney in close association with the peritubular capillary and proximal convoluted tubule. It is also produced in perisinusoidal cells in the liver. Liver production predominates in the fetal and perinatal period; renal production predominates in adulthood. It is homologous with thrombopoietin.

Exogenous erythropoietin, recombinant human erythropoietin (rhEPO), is produced by recombinant DNA technology in cell culture and are collectively called erythropoiesis-stimulating agents (ESA): two examples are epoetin alfa and epoetin beta. ESAs are used in the treatment of anemia in chronic kidney disease, anemia in myelodysplasia, and in anemia from cancer chemotherapy. Risks of therapy include death, myocardial infarction, stroke, venous thromboembolism, and tumor recurrence. Risk increases when EPO treatment raises hemoglobin levels over 11 g/dL to 12 g/dL: this is to be avoided.

EPO is highly glycosylated (40% of total molecular weight), with half-life in blood around 5 h. EPO's half-life may vary between endogenous and various recombinant versions. Additional glycosylation or other alterations of EPO via recombinant technology have led to the increase of EPO's stability in blood (thus requiring less frequent injections).

Erythropoietin is an essential hormone for red blood cell production. Without it, definitive erythropoiesis does not take place. Under hypoxic conditions, the kidney will produce and secrete erythropoietin to increase the production of red blood cells by targeting CFU-E, proerythroblast and basophilic erythroblast subsets in the differentiation. Erythropoietin has its primary effect on red blood cell progenitors and precursors (which are found in the bone marrow in humans) by promoting their survival through protecting these cells from apoptosis, or cell death.

Erythropoietin is the primary erythropoietic factor that cooperates with various other growth factors (e.g., IL-3, IL-6, glucocorticoids, and SCF) involved in the development of erythroid lineage from multipotent progenitors. The burst-forming unit-erythroid (BFU-E) cells start erythropoietin receptor expression and are sensitive to erythropoietin. Subsequent stage, the colony-forming unit-erythroid (CFU-E), expresses maximal erythropoietin receptor density and is completely dependent on erythropoietin for further differentiation. Precursors of red cells, the proerythroblasts and basophilic erythroblasts also express erythropoietin receptor and are therefore affected by it.

### V. Cell Engineering

In certain embodiments, cells are transformed or engineered with expression cassettes to express a transcription modulating protein. Provided herein are such expression vectors which contain one or more nucleic acids encoding transcription modulating proteins. Additional elements such as target genes and/or sgRNAs and control sequences therefor may also be introduced into cells.

Expression requires that appropriate signals be provided in the vectors and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2b, HSP70, proliferin, tumor necrosis factor, and/or thyroid stimulating hormone a gene. In some embodiments, the inducer is phorbol ester (TFA), heavy metals, glucocorticoids, poly(rI)x, poly(rc), EIA, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Of particular interest are muscle specific promoters. These include the myosin light chain-2 promoter, the α-actin promoter, the troponin 1 promoter, the Na⁺/Ca²⁺ exchanger promoter, the dystrophin promoter, the α7 integrin promoter, the brain natriuretic peptide promoter and the αB-crystallin/small heat shock protein promoter, α-myosin heavy chain promoter and the ANF promoter. In some embodiments, the muscle specific promoter is the CK8 promoter. The CK8 promoter has the following sequence (SEQ ID NO. 874):

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

B. Delivery of Expression Vectors

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation. In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

The adenoviruses of the disclosure are replication defective, or at least conditionally replication defective. The adenovirus may be of any of the 42 different known serotypes or subgroups A-F. Adenovirus type 5 of subgroup C is the preferred starting material in order to obtain the conditional replication-defective adenovirus vector for use in the present disclosure.

As stated above, the typical vector according to the present disclosure is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors, or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and therapeutic potential as in vivo gene transfer vectors.

Adenovirus vectors have been used in eukaryotic gene expression and vaccine development. Animal studies suggested that recombinant adenovirus could be used for gene therapy. Studies in administering recombinant adenovirus to different tissues include trachea instillation, muscle injection, peripheral intravenous injections and stereotactic inoculation into the brain.

The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses may be used, in which biotinylated antibodies against a retroviral envelope protein and against a specific cell receptor are used. The antibodies are coupled via the biotin components by using streptavidin.

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes. Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome.

A particular type of retrovirus is a lentivirus. The virus contains a reverse transcriptase molecule found to perform transcription of the viral genetic material upon entering the cell. Within the viral genome are RNA sequences that code for specific proteins that facilitate the incorporation of the viral sequences into the host cell genome. The “gag” gene codes for the structural components of the viral nucleocapsid proteins: the matrix (MA/p17), the capsid (CA/p24) and the nucleocapsid (NC/p7) proteins. The “pol” domain codes for the reverse transcriptase and integrase enzymes. Lastly, the “env” domain of the viral genome encodes for the glycoproteins and envelope on the surface of the virus.

There are multiple steps involved in the infection and replication of a lentivirus in a host cell. In the first step the virus uses its surface glycoproteins for attachment to the outer surface of a cell. More specifically, lentiviruses attach to the CD4 glycoproteins on the surface of a host's target cell. The viral material is then injected into the host cell's cytoplasm. Within the cytoplasm, the viral reverse transcriptase enzyme performs reverse transcription of the viral RNA genome to create a viral DNA genome. The viral DNA is then sent into the nucleus of the host cell where it is incorporated into the host cell's genome with the help of the viral enzyme integrase. From now on, the host cell starts to transcribe the entire viral RNA and express the structural viral proteins, in particular those that form the viral capsid and the envelope. The lentiviral RNA and the viral proteins then assemble and the newly formed virions leave the host cell when enough are made.

One method of gene therapy involves modifying a virus to act as a vector to insert beneficial genes into cells. Unlike other retroviruses, which cannot penetrate the nuclear envelope and can therefore only act on cells while they are undergoing mitosis, lentiviruses can infect cells whether or not they are dividing. Many cell types, like neurons, do not divide in adult organisms, so lentiviral gene therapy is a good candidate for treating conditions that affect those cell types.

Some experimental applications of lentiviral vectors have been done in gene therapy in order to cure diseases like diabetes mellitus, murine hemophilia A, prostate cancer, chronic granulomatous disease, and vascular diseases.

Therapy requires manipulation of the lentivirus genes and structure for delivery of specific genes to alter the course of the disease. Parts of the viral genome must be removed so that the virus can't replicate itself. It is replaced with a gene to permanently incorporate into the host cell's genome using genetically modified virus.

HIV-derived lentiviral vectors have been used for introducing libraries of complementary DNAs, short hairpin RNAs, and cis-regulatory elements into many targets, including embryonic stem cells.

Other viral vectors may be employed as expression constructs in the present disclosure. Vectors derived from viruses such as vaccinia virus adeno-associated virus (AAV) and herpesviruses may be employed. They offer several attractive features for various mammalian cells.

In embodiments, the AAV vector is replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

Several non-viral methods for the transfer of expression constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000™ is widely used and commercially available.

In certain embodiments, the liposome may be complexed with a hemagglutinating virus (HVJ) to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### VI. Kits

In still further embodiments, cells and expression constructs for use in accordance with the present disclosure can be provide in kits for use with the methods described above. The kits may thus comprise, in suitable container means, one or more cells, one or more expression constructs, salicylic acid, and other optional reagents. The components of the kits may be packaged either in aqueous media or in lyophilized form. The kits may also contain instructions for use.

The container means of the kits will generally include at least one vial, test tube, flask, bottle, syringe or other container means, into which the reagent may be placed, or preferably, suitably aliquoted. The kits of the present disclosure will also typically include a means for containing the reagents and containers in close confinement for commercial sale. Such commercial packages may include injection or blow-molded plastic containers into which the desired containers are retained.

### VII. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Results

Establishing controls for the localization assay. It has been proposed that the C-terminal transactivation domain (TAD) of NPR1, amino acids 513-593, harbors the salicylic acid binding domain and the nuclear localization signal. Furthermore, the recombinant protein containing only amino acids 513-593 has been demonstrated to bind directly to salicylic acid with an affinity of ˜1.5 μM. The inventors' hypothesized that, similar to estrogen receptors, the NPR1-TAD was sufficient for the nuclear translocation of passenger proteins. They elected to test this using a heterologous expression in mammalian cells under the reasoning that any other plant specific, NPR1 associated accessory proteins (e.g., NPR3/NPR4) needed for translocation are likely absent in mammalian cells.

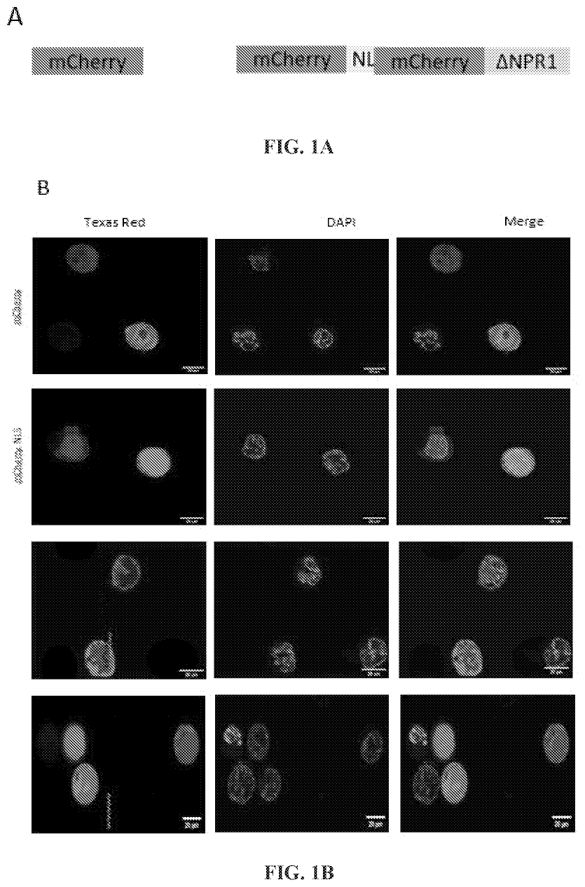

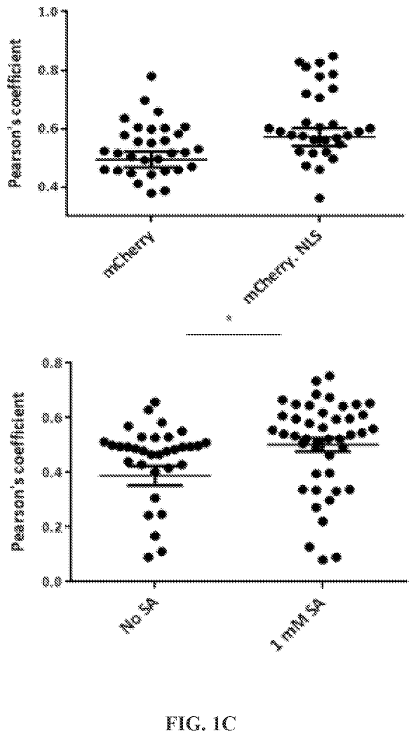

To establish negative and positive controls for nuclear localization, the inventors transfected mCherry with (mCherry.NLS) and without an NLS sequence into HEK293 cells. As expected, imaging using confocal microscopy confirmed that the mCherry protein was distributed both in the nucleus and cytoplasm whereas cells expressing mCherry.NLS showed predominant nuclear localization (FIG. 1B). The inventors quantified the degree of localization by performing ImageJ analysis and calculating the degree of localization as represented by Pearson's coefficient. FIG. 1C shows that mCherry.NLS has higher protein level resident in the nucleus as compared to mCherry, which does not possess the NLS sequence necessary for nucleocytoplasmic translocation. Hence, the inventors investigated the effect of SA on the localization of mCherry.ΔNPR1 in HEK293T mammalian cells. Thirty random cells were imaged before and 24 h after addition of SA at 1 mM final concentration. In the absence of SA, mCherry.ΔNPR1 is significantly concentrated in the cytoplasm area (FIG. 1B). However, a significant increase in Pearson coefficient 24 h after addition of SA suggests that only the ΔNPR1, which is believed to be the domain playing a key role in SA binding, is sufficient to activate the nucleocytoplasmic localization of the whole fusion protein (FIG. 1C).

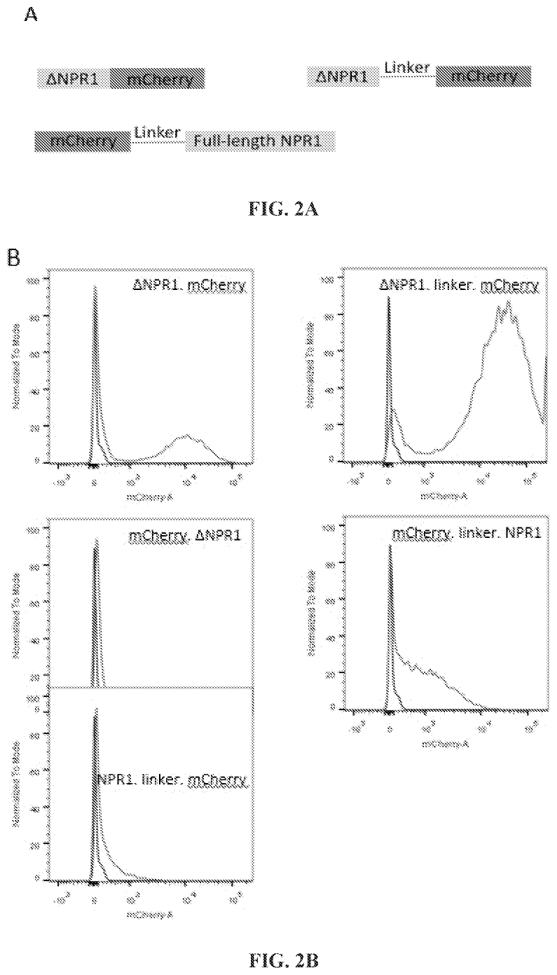

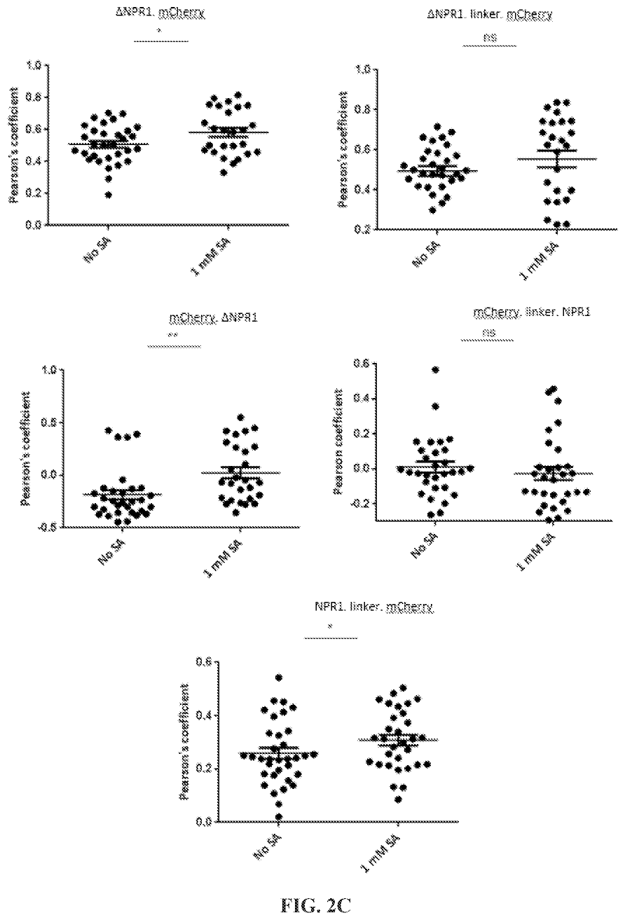

Investigating SA-dependent localization in other variants of NPR1 protein. The inventors next examined SA-dependent localization of different NPR1 protein variants by N- or C-terminal fusion of the A513 domain or the full-length NPR1 to mCherry reporter protein (FIG. 2A). Flexible Glycine-Serine linkers were inserted between the cargo and NPR1 variants to allow for mobility and flexibility of the connecting domains. Full-length NPR1 or ΔNPR1 were fused to mCherry with or without linker as demonstrated by FIG. 2A. Plasmids were stably transfected into HEK293T cells and grown for two weeks before being sorted for mCherry+ cells. Histograms representing the sorted populations and negative un-transfected control are illustrated in FIG. 2B.

Next, mCherry localization was tested in the stable cell lines in the absence and presence of SA. The results suggest that mCherry.ΔNPR1, ΔNPR1.mCherry, and NPR1.Linker.mCherry cells show SA-dependent nucleocytoplasmic localization as evidenced by greater Pearson's coefficient in 1 mM SA conditions versus at its absence. However, the inventors did not observe this effect in ΔNPR1.Linker.mCherry and mCherry.Linker.NPR1 constructs. See FIG. 2C.

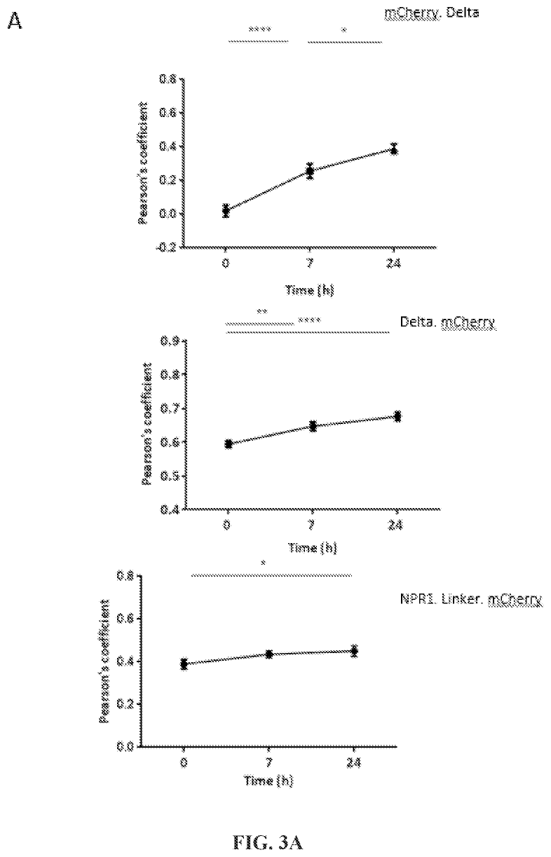

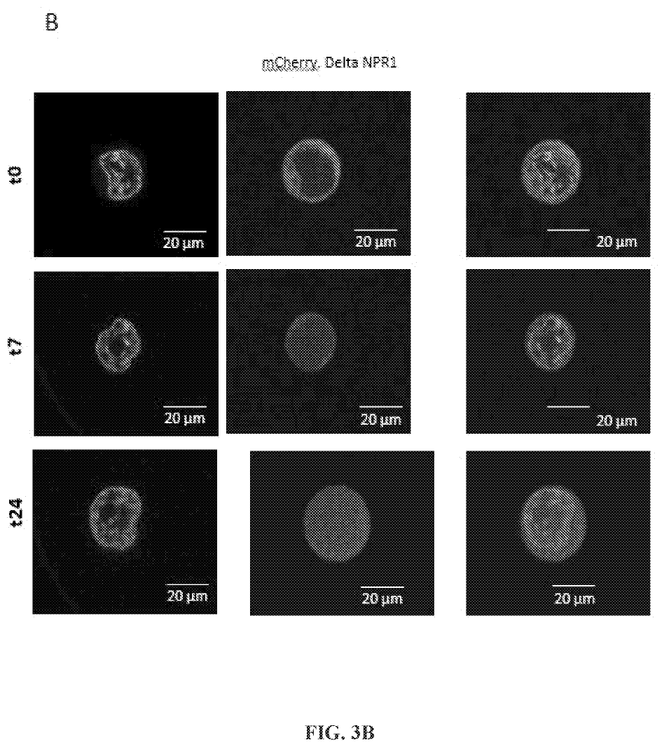

Single-cell SA-dependent protein localization using mesh microarray system. To demonstrate the time-dependence of localization of the fusion proteins that responded to SA in bulk assay, the inventors performed the experiment at the single-cell level using a mesh microarray system. The micromesh array contains nanoliter wells that allowed the inventors to dynamically image protein translocation within the same individual cells. The inventors followed the kinetics of translocation after 6 h and after 24 h. Cells that stably express mCherry. ΔNPR1, NPR1.linker.mCherry, and ΔNPR1.mCherry were loaded on the mesh with 100 μm-deep wells after nucleic acid staining and imaged using a 100× objective on the confocal microscope. Cells were imaged 7 h and 24 h after addition of salicylic acid to the final concentration of 2.5 Mm (FIG. 3B). Data from image analysis further supports that mCherry.ΔNPR1, NPR1.linker.mCherry, and ΔNPR1.MCherry proteins are shuttled to nucleus and show time-dependent increase in their degree of localization (FIG. 3A).

Summary From a biotechnological perspective, the NPR1-TAD is attractive as a SA mediated nuclear translocator with the best translocator, mCherry-NPR1-TAD performing nuclear translocation as efficiently as widely utilized the estrogen receptor alpha system. There are several advantages to this system. The small size of the TAD minimizes the metabolic load of protein expression. Second, since it is of non-mammalian origin, it is likely immunogenic but again the small size decreases the number of available epitopes. Third, since it is not of microbial origin, it is not likely to be compromised by pre-existing immunity. From an application standpoint, when fused at the C-terminus of the passenger protein, the basal state is strong nuclear exclusion and hence might be appropriate for DNAse based kill switches in adoptive cell therapy or Cas9 based inducible editors. When fused at the N-terminus of the passenger protein, the induced state is strong nuclear localization and hence this might be attractive for transactivation of gene expression. The availability of small protein domains like the NPR1-TAD that can facilitate SA mediated nuclear translocation in mammalian cells has strong potential for translational applications within living organisms.

### Example 2—Materials and Methods (for Examples 3-4)

Molecular cloning using pcDNA3.4 vector. The inventors obtained the gene fragments coding for mCherry, mCherry-NLS, and mCherry-NPR1-TAD by PCR. For the construction of the mCherry-NLS, the NLS sequence from the simian vacuolating virus (SV40) was fused to the mCherry gene at the C-terminus. The gene fragment coding for NPR1 was purchased from Integrated DNA Technologies. The plasmid containing the mCherry gene was a kind gift from Dr. Xinping Fu (University of Houston). The inventors amplified the gene encoding mCherry-NPR1-TAD by PCR using primers designed to encode a Hiss tag (3′), in addition to the restriction enzyme recognition sites (5′ and 3′). Primers for mCherry and mCherry-NLS constructs were designed without the Hiss tag. Genetic fusion of mCherry to NPR1-TAD was accomplished via overlap extension PCR (OE-PCR). The inventor digested the PCR products and pcDNA3.4 using Bsu36I-HF and AgeI-HF at 37° C. for 3 h and ligated them using T4 DNA ligase at 16° C. overnight. They transformed the plasmids into E. coli MC1061 cells by electroporation and verified the sequences by standard Sanger sequencing (Genewiz, USA). Next, single colonies for each of the mCherry, mCherry-NLS, and mCherry-NPR1-TAD constructs were inoculated in 100 ml of Lysogeny broth (LB) medium supplemented with 200 μg/ml ampicillin in separate flasks. The inventors grew the cells at 37° C. overnight in an orbital shaker. Plasmid DNAs were isolated using QIAGEN plasmid maxi kit and QIAvac 24 plus vacuum manifold (Qiagen Inc., USA).

Molecular cloning using dCAS9_VP64_GFP lentiviral backbone. The inventors used Gibson assembly for the cloning of the constructs mCherry-NPR1-TAD, NPR1-TAD-mCherry, mCherry-linker-NPR1, and NPR1-linker-mCherry using the backbone of dCAS9_VP64_GFP (Addgene, plasmid #61422). The linker used in these constructs was (SGGG)₁(SGGGG)₂. The constructs were transformed by heat shock into E. coli E cloni chemical competent cells following the protocol described by the manufacturer (Lucigen, USA). The inventors isolated the plasmids and confirmed their sequence by Sanger sequencing.

Transfection into HEK293T cells. The inventors used low-passage HEK293T cells with greater than 90% viability for transient transfection of the plasmid constructs using Lipofectamine LTX reagent (Thermo Fisher Scientific). The cells were trypsinized and counted using trypan blue (STEMCELL Technologies, USA). 5×10⁵ cells were seeded into a well of 6-well plate the day before transfection in 3 mL R10 (RPMI-1640 supplemented with 10% FBS and L-glutamine) growth medium to 50-80% confluency. On the day of transfection, 2 μg of the plasmid along with 2 μl PLUS reagent from the Lipofectamine LTX transfection kit (Invitrogen, CA) was added to 200 μl of the opti-MEM media (Invitrogen, USA). Next, 4 μl of the Lipofectamine LTX reagent was diluted in 200 μl of the same media, and each reaction mix was incubated separately. After 5 min, the two tube contents were mixed to allow the DNA-Lipofectamine complex to form. After 30 min of incubation, the mixture was added to the cells, and 4 h later the media was replaced with 3 mL of fresh R10.

Generation of the stable cell line. 5×10⁶ of HEK293 cells were seeded into a T25 flask with 5 mL DMEM F12 media the day before transfection. On the day of transfection, 6 μg of the target plasmid, 5 μg of the psPAX plasmid, and 3 μg of the MD2G plasmid were transfected into HEK293 cells using Lipofectamine transfection protocol. One day after transfection, the media was removed and replaced with 10 ml of fresh R10 medium. The inventors harvested the supernatant from the cells after 72 h and concentrated the supernatants using Amicon Ultra-15 filters. These viral particles were used to infect 6×10⁶ HEK293 cells. Next, the mCherry-expressing HEK293 cells were sorted (FACSAria Fusion, MDACC) one week after transduction. Sorted cells were propagated using R10 medium and used for subsequent experiments.

Staining and microscopy. Cells expressing the protein of interest were harvested by spinning down at 350×g for 5 min and washed with 1×PBS three times. 1×10⁶ cells were resuspended in 1 ml of 1×PBS, and 10 μg/ml of the Hoechst 33342 dye (Thermo Fisher Scientific, USA) was added to the cells and incubated at 37° C. for 20 min. Next, 4×10⁵ cells were loaded into a 35-mm petri dish, and mCherry-positive cells were imaged using a 100×(oil) 1.49 NA objective on Al/TiE inverted confocal microscope (Nikon Instruments Inc., USA). Image analysis was performed on at least thirty cells that were positive for both mCherry protein and Hoechst dye. N/C/D analysis was performed by visualization. Translocation was considered to be “predominantly in the nucleus” (N) if no mCherry signal was seen in other cellular compartments. The same decision-making trend was applied to “predominantly in the cytoplasm” (C) and “distributed all over the cell” (D) conditions. To track the localization of single-cells, the inventors loaded 100 μl of stained cells at a density of 1×10⁶/ml into a micromesh array with a 50 μm depth size (microsurfaces, Australia). The Pearson's co-localization coefficient was calculated using JaCop plugin on ImageJ.

SA dose optimization assay. SA was added to cells at final concentrations of 1 μM, 10 μM, 100 μM, 500 μM, 1 mM and 2.5 mM. Cells were imaged before addition of SA and 24 hours after addition of SA. The relative change in average PCC at different concentrations with respect to no addition of SA was calculated using the formula (X_{c}−X₀)±\sqrt{(SEM_{c}²+SEM₀²)}, where X_{C} and X₀ are mean PCC values and SEM_{C} and SEM₀ are corresponding standard error of the mean(SEM) at SA concentration C and 0 respectively. The relative change in PCC was plotted against SA concentration and linear regression was performed in Graphpad Prism.

Reversibility assay. The inventors recorded an initial image of the cells in the absence of SA. Next, they added SA at a final concentration of 2.5 mM, and the reversibility potential of the NPR1 variants was determined by monitoring the co-localization for up to 30 h after the addition of SA. At this time, the media containing SA was carefully decanted and replaced with fresh R10 media. The cells were placed in the incubator for 18 more hours (48 h from time zero) and images were captured using a confocal microscope.

Structure prediction of AtNPR1. The structure of AtNPR1 was predicted using the Phyre2 web portal for protein modeling, prediction and analysis (Kelley el al., 2015). The predicted model was visualized and edited using the EzMol interface (Reynolds et al., 2018).

### Example 3—Results

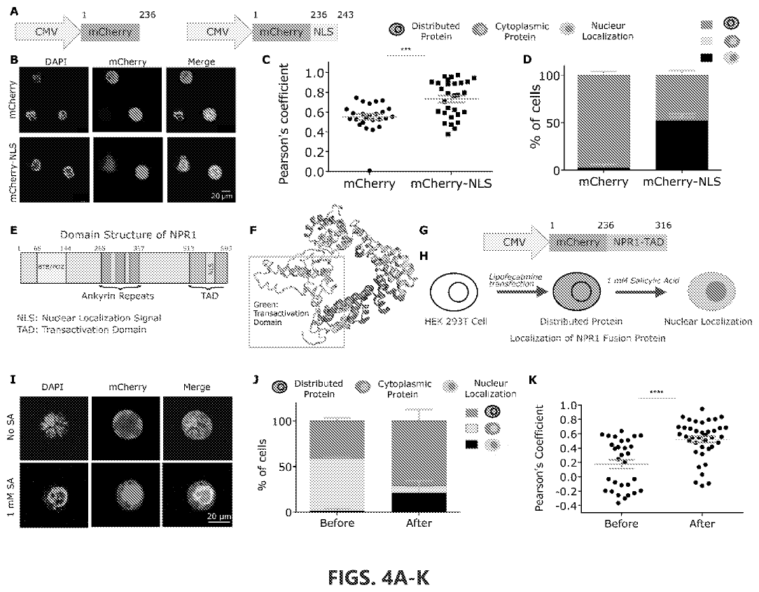

NPR1-TAD is sufficient for translocation of mCherry. To establish controls and determine the dynamic range for nuclear localization, the inventors cloned mCherry and mCherry fused to a C-terminal nuclear localization signal (mCherry-NLS) into a pcDNA based plasmid. The plasmids were individually transfected into HEK293 cells (FIG. 4A). The cells were stained with the nuclear stain, DAPI, and visualized by fluorescent confocal microscopy (FIG. 4B). The inventors quantified the degree of localization by subcategorizing cells expressing cargo mCherry as N (predominantly in the nucleus), C (predominantly in the cytoplasm), and D (distributed all over the cell). As expected, 97% of HEK293 cells transfected with mCherry showed diffuse staining indicating that the protein was present both in the cytoplasm and the nucleus (FIGS. 4B and 4D). By contrast, 52% of cells transfected with mCherry-NLS showed predominant nuclear localization (FIGS. 4B and 4D). The inventors quantified the degree of nuclear co-localization by computing Pearson's correlation coefficient (PCC) (FIG. 4C). PCC values close to one imply nuclear localization of mCherry while a value close to −1 implies nuclear exclusion of mCherry (FIGS. S2A-C). These results also confirmed that cells expressing mCherry-NLS showed an enrichment of the protein in the nucleus compared to cells expressing mCherry.

It has been proposed that the TAD domain of Arabidopsis thaliana NPR1 (AtNPR1), amino acids 513-593, harbors the SA-binding domain and the NLS fragment (FIG. 4E) (Wu et al., 2012). The NLS is of AtNPR1 is an 18-aa long peptide located in between amino acids 537-554 with the complete sequence of KRLQKKQRYMEIQETLKK (UniProtKB-P93002 (NPR1-ARATH)). Although the structure of AtNPR1 is not available, the predicted structure based on modeling indicated that NPR1 comprises sets of helix bundles and that the TAD is a part of one such bundle (FIG. 4F). The inventors thus hypothesized that the NPR1-TAD is sufficient for the ligand-induced nuclear translocation of passenger proteins. They tested this hypothesis using a heterologous expression platform in mammalian cells with the reasoning that any other plant-specific NPR1 associated accessory proteins needed for translocation are absent in mammalian cells. Accordingly, the inventors cloned the mCherry-NPR1-TAD fusion protein into the same pcDNA backbone (FIG. 4G). The inventors transiently transfected the plasmid into HEK293 cells and imaged the localization of mCherry before and after the addition of 1 mM SA (Wu et al., 2012) (FIG. 4H). In the absence of SA, 57% of the cells expressing mCherry-NPR1-TAD showed predominant cytoplasmic staining (FIGS. 4I-J). 24 h after the addition of 1 mM SA, only 8% of cells showed predominant cytoplasmic staining (FIGS. 4I-J). This change was also reflected in the nuclear localization of the proteins within the same cells. The frequency of cells expressing mCherry in the nucleus (D & N staining) increased from 43% to 92% (FIG. 4J). The inventors quantified the overlap of the mCherry signal with the nuclear stain using PCC. Consistent with the subcellular classification, 57% of the cells showed a negative correlation in the absence of SA, indicative of cytoplasmic expression. In the presence of SA 92% of cells showed a significant nuclear correlation (FIG. 4K, 0.18±0.03 vs 0.56±0.09, p-value<0.0001). Collectively, these results using transient transfections, established that the NPR1-TAD is sufficient for SA induced nuclear translocation of mCherry in human cells. The magnitude of cells with increased nuclear localization upon the addition of ligand compares favorably to the tamoxifen inducible estrogen receptor alpha system³.

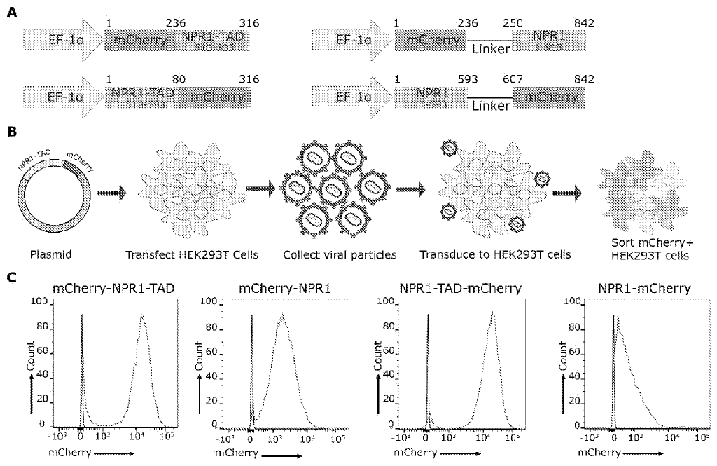

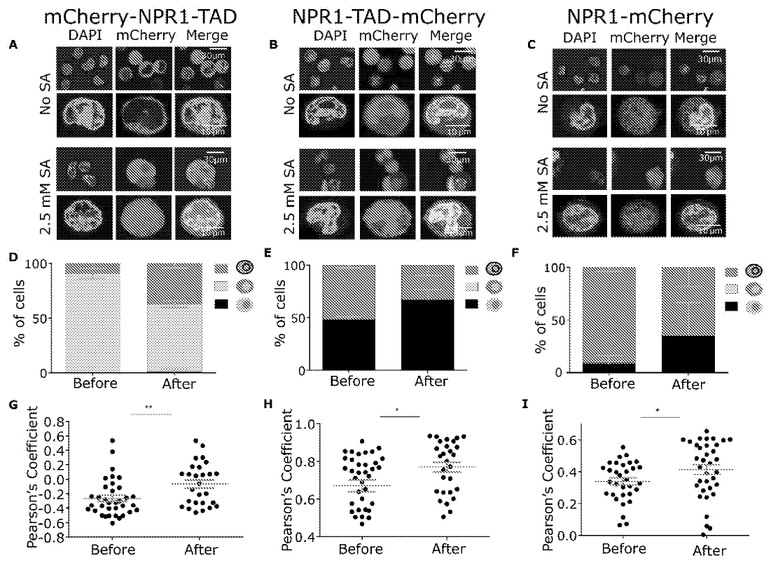

Differential subcellular localization of NPR1 fusion proteins. In initial experiments, the inventors fused the NPR1-TAD at the C-terminus of mCherry since this is consistent with the localization of TAD within NPR1. Having established that the NPR1-TAD can mediate the nuclear translocation of mCherry, the next aim was to systematically investigate whether (a) TAD can function at both N and C termini, and (b) if the translocation mediated by full-length NPR1 behaved differently to the translocation mediated by the NPR1-TAD. Accordingly, the inventors designed four separate constructs with mCherry as the reporter protein: mCherry-NPR1-TAD, NPR1-TAD-mCherry, mCherry-NPR1, and NPR1-mCherry (FIG. 5A). Flexible Glycine-Serine linkers were inserted in constructs harboring full-length NPR1 to allow for the mobility of the connecting domains (Chen et al., 2013). The inventors cloned the constructs downstream of the EF-1α promoter, transduced them into HEK293 cells, and flow-sorted based on mCherry fluorescence to generate stable cell lines (FIGS. 5B-C).

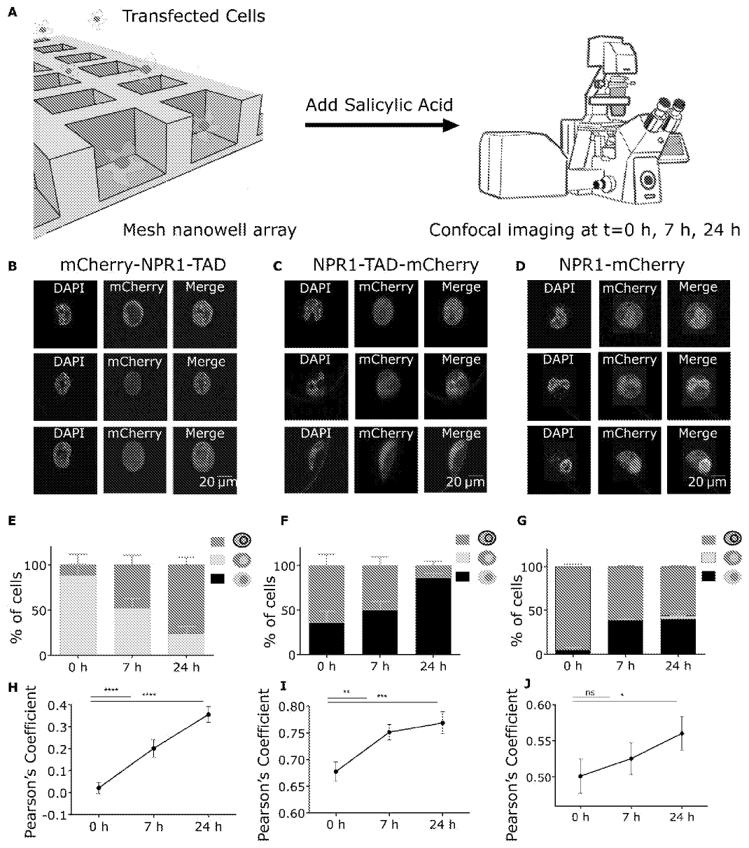

To study potential nucleocytoplasmic shuttling, the inventors quantified the localization of mCherry in these stable cell lines in the presence and absence of SA using confocal microscopy. As a control, they cultured the cells without addition of SA for 24 hours and verified that the localization of mCherry was invariant with time (FIGS. S3A-C). Next, they performed SA dose optimization studies for cells stably expressing mCherry-NPR1-TAD, NPR1-TAD-mCherry and NPR1-mCherry at concentrations varying from 1 μM-2.5 mM. The inventors observed a linear correlation between increase in average PCC values and SA concentration for all three constructs (FIGS. S4A-F). Although they used SA at 1 mM concentration in transient transfection experiments, they increased the final SA concentration to 2.5 mM based on dose optimization experiments (FIGS. S4A-F). Consistent with the data the inventors obtained with transient transfections, only 4% of cells expressing mCherry-NPR1-TAD showed nuclear localization (D & N) in the absence of SA (FIGS. 6A, 6D and 6G). Upon the addition of SA, 32% of cells showed nuclear localization of mCherry (FIG. 6D) and this was also reflected in significant increase in nuclear colocalization by PCC (−0.26±0.04 vs −0.06±0.01, p-value=0.006). By comparison, 24% of HEK293 cells expressing the construct with full-length NPR1 at the C-terminus, mCherry-NPR1, showed nuclear localization (D & N) in the absence of SA (FIG. S1A) and this number was unaltered by the addition of SA (FIG. S1B). PCC was also consistent with a lack of change in protein localization in these cells upon the addition of SA (FIG. S1C, −0.03±0.01 vs −0.08±0.02). Collectively these results showed that while both NPR1 and NPR1-TAD when fused to the C-terminus of mCherry facilitate efficient nuclear exclusion, only NPR1-TAD is capable of nuclear translocation in the presence of SA.

The inventors next investigated the two constructs with NPR1 domains at the N-terminus. HEK293 cells expressing NPR1-TAD-mCherry showed a different distribution of mCherry proteins compared to mCherry-NPR1-TAD. 100% of the cells showed nuclear expression (D & N) of mCherry in the presence or absence of SA (FIGS. 6B, 6E and 6H). 47% of the cells had predominant nuclear localization in the absence of SA and the addition of SA increased this frequency to 73%. Thus, the frequency of cells showing only nuclear localization of mCherry increased in the presence of SA and this also led to the significantly increased nuclear colocalization by PCC (0.67±0.11 vs 0.77±0.14, p-value=0.018). Similar to the NPR1-TAD-mCherry construct, 100% of HEK cells expressing NPR1-mCherry showed nuclear expression (D & N) of mCherry in the presence or absence of SA (FIGS. 6C, 6F and 6I). 7% of these cells had predominant nuclear localization in the absence of SA and the addition of SA increased this frequency to 41%. Thus, the frequency of cells showing predominant nuclear localization of mCherry increased in the presence of SA and this also led to the significantly increased nuclear colocalization by PCC (FIG. 6I, 0.34±0.06 vs 0.45±0.07, p-value=0.026). Collectively these results showed that both NPR1 and NPR1-TAD when fused to the N-terminus of mCherry facilitate basal nuclear translocation in the absence of SA but the translocation is significantly increased in response to SA. The major difference between the two constructs was that NPR 1-mCherry showed lower nuclear colocalization compared to NPR1-TAD-mCherry both in the presence and absence of SA (FIGS. 6E and 6F).

In aggregate, data from all four of these constructs illustrated that regardless of responsiveness to SA, C-terminal NPR1 fusions facilitate cytoplasmic expression (cells are predominantly either C or D) whereas N-terminal NPR1 fusions facilitate nuclear expression (cells are predominantly either N or D).

Single-cell SA-dependent protein localization using mesh microarray system. Since the inventors established that three constructs; mCherry-NPR1-TAD, NPR1-TAD-mCherry, and NPR1-mCherry showed translocation of mCherry mediated by SA, they prioritized these for further characterization. The inventors utilized a mesh microarray system to understand the kinetics of translocation at the single-cell level and to enable tracking of the same individual cells. The micromesh array contains nanoliter wells that allowed us to image protein translocation dynamically (FIG. 7A).

Cells that stably express mCherry-NPR1-TAD, NPR1-mCherry, and NPR1-TAD-mCherry were loaded on the mesh with 100 μm depth and imaged using a confocal microscope. Cells were imaged 7 h and 24 h after the addition of SA (FIGS. 7B-D). Single-cell tracking experiments confirmed the previously observed behaviors with each of the constructs. cells expressing mCherry-NPR1-TAD transitioned from predominantly cytoplasmic expression (C at 0 h: 88%, C at 7 h: 52%, and C at 24 h: 24%) to more diffuse expression (D at 0 h:12%, D at 7 h: 48%, and D at 24 h: 76%) (FIG. 7E). These results are also reflected the time-dependent increase in PCC upon addition of SA (FIG. 7H). The inventors observed that cells expressing the two N-terminal fusions, NPR1-mCherry, and NPR1-TAD-mCherry, showed an increased frequency of cells expressing mCherry predominantly in the nucleus in response to the addition of SA (FIGS. 7F-G). This was reflected in a time-dependent increase in PCC upon the addition of SA (FIGS. 7I-J). The magnitude of protein translocation was most pronounced for mCherry-NPR1-TAD and least for NPR1-TAD-mCherry. Collectively, these results established that the translocation of the NPR1 fusion proteins is completed within 24 h.

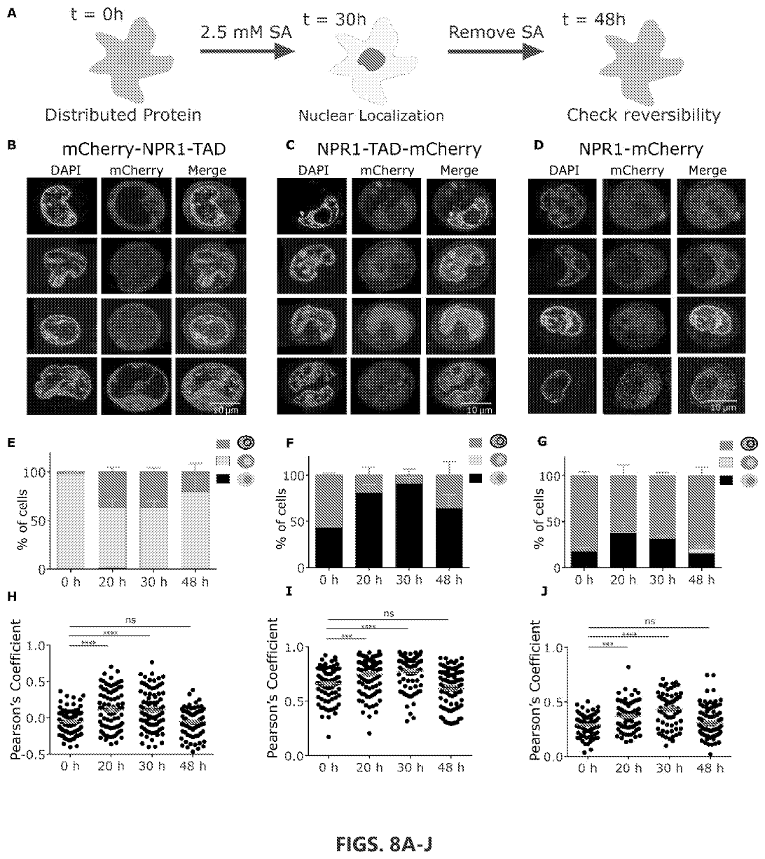

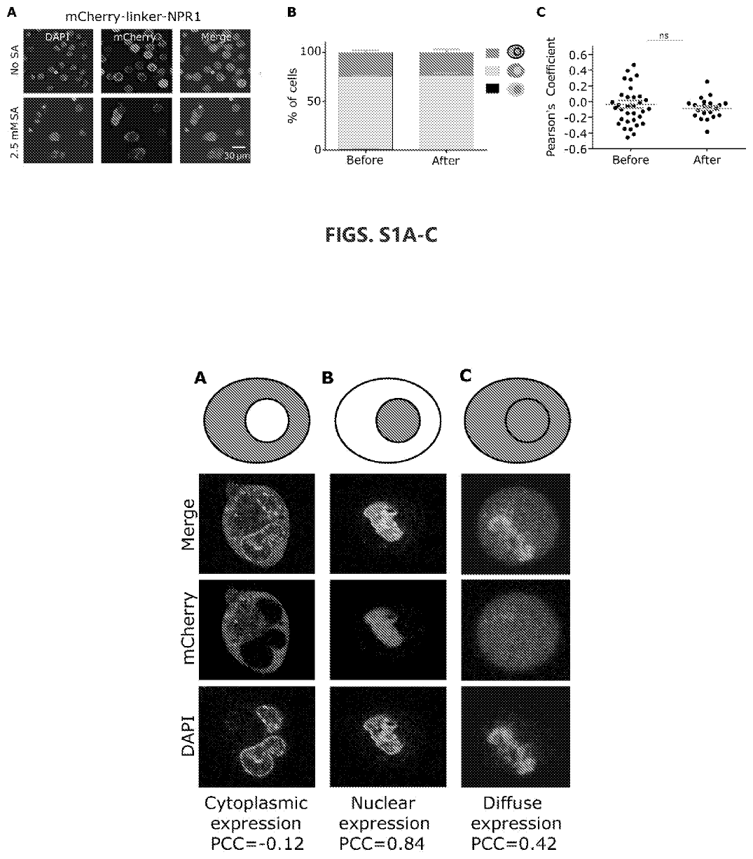

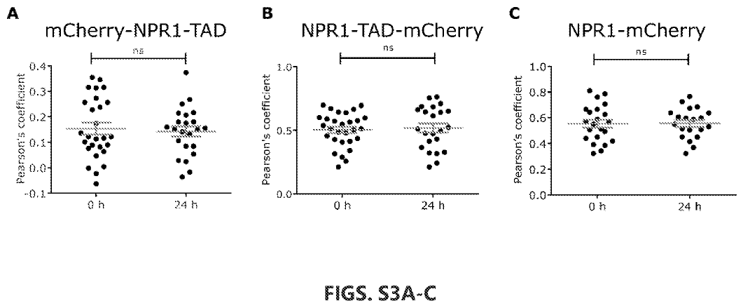

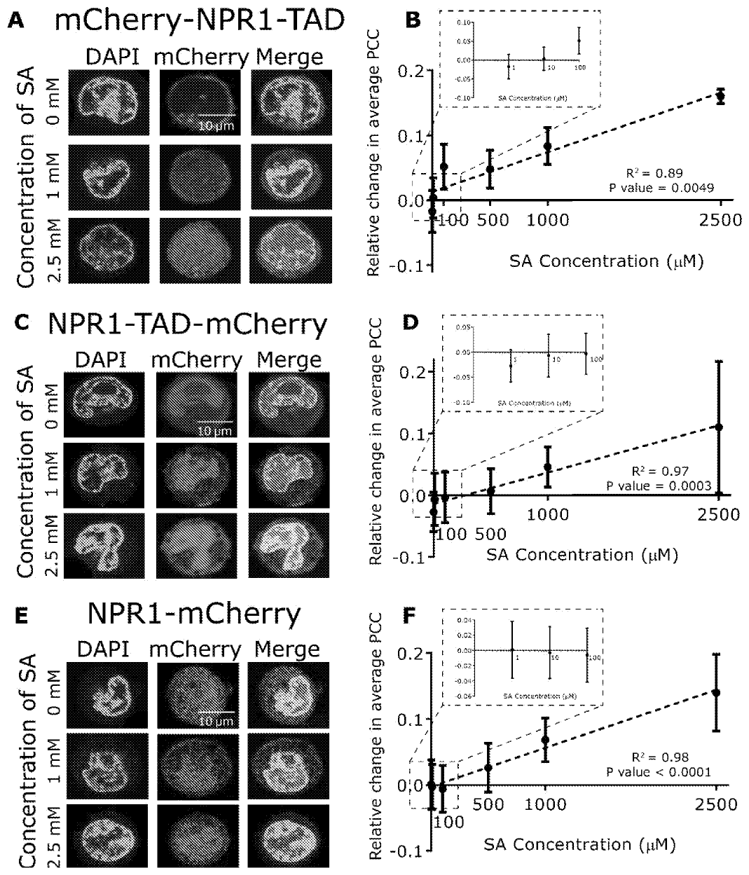

Reversibility of the NPR1 fusion proteins. After extensively characterizing the SA mediated nucleocytoplasmic shuttling of mCherry constructs, the inventors next investigated whether removing SA would reverse the nuclear translocation of mCherry. They examined the reversibility of mCherry localization at three stages: (a) basal, before the addition of SA (0 h); (b) induced, at 20 h and 30 h after the addition of SA; and (c) reversed, at 48 h, wherein after 30 h the media containing SA was removed and replaced with SA free media (FIG. 8A). After the removal of SA, the cells expressing mCherry-NPR1-TAD transitioned from diffuse (D=37%, C=63% at 30 h) to cytoplasmic localization (D=20%, C=80% at 48 h) (FIGS. 8B and 8E). Tracking the PCC confirmed nuclear translocation of mCherry from the basal to the induced states (−0.05±0.005 vs 0.11±0.01, p-value<0.0001) and reversal after the withdrawal of SA (FIG. 8H, −0.05±0.005 vs −0.05±0.005).

The inventors observed similar behavior with both constructs expressing NPR1 or NPR1-TAD at the N-terminus. The frequency of cells expressing NPR1-TAD-mCherry displaying predominant nuclear localization increased upon the addition of SA (43% to 91%, FIG. 8F). Upon the removal of SA, the percentage of cells exhibiting predominant nuclear localization reduced to 64% at 48 h (FIG. 8F). This behavior was also captured in the PCC data (FIG. 8I, 0.66±0.07 at t=0 h vs 0.77±0.08 at t=30 h, p-value<0.0001 and 0.66±0.07 at t=0 h vs 0.62±0.07 at t=48 h, p-value=0.26). Although the frequency of cells with predominant nuclear localization was lower with cells expressing NPR1-mCherry compared to NPR1-TAD-mCherry, the inducible and reversible behavior was largely conserved (FIG. 8J, 0.29±0.03 at t=0 h vs 0.43±0.05 at t=30 h p-value<0.0001 and 0.29±0.03 at t=0 h vs 0.31±0.03 at t=48 h, p-value=0.63). Consistent with single-cell tracking experiments, protein translocation and reversibility was most pronounced for mCherry-NPR1-TAD and least for NPR1-TAD-mCherry. Collectively, these data suggest that translocation mediated by SA is both inducible and reversible.

TABLE 1
Summary of results obtained from SA mediated translocation studies in HEK293 cells.

| | | | | | | | Cys82 and | | |
|---|---|---|---|---|---|---|---|---|---|
| | | | Subcellular | | | NPR1 has a | Cys216 of | | |
| | Size | Predicted | localization | | SA mediated | free N or C- | NPR1 | | |
| Construct | (kDa) | transport | No SA | With SA | change? | term? | present? | Reversible? | Comment |
| mCherry- | ~93 | Active | C = 100 | C = 100 | No translocation | C | Yes | N/A | Strong nuclear |
| NPR1 | | | PCC −0.03 | PCC −0.08 | | | | | exclusion in the |
| | | | | | | | | | absence of SA: no |
| | | | | | | | | | translocation. |
| mCherry- | ~36 | Passive/ | C = 98 | C = 63 | 1 Diffuse/nuclear | C | No | Yes | Strong nuclear |
| NPR1-TAD | | active | D = 2 | D = 37 | | | | C = 80, | exclusion in the |
| | | | PCC −0.05 | PCC 0.11 | | | | D = 20 | absence of SA |
| | | | | | | | | PCC −0.05 | |
| NPR1- | ~36 | Passive/ | D = 57 | D = 9 | 1 Nuclear | N | No | Yes | Strong nuclear |
| TAD- | | active | N = 43 | N = 91 | | | | N = 64, | localization |
| mCherry | | | PCC 0.66 | PCC 0.77 | | | | D = 36 | after the addition |
| | | | | | | | | PCC 0.62 | of SA |
| NPR1- | ~93 | Active | D = 82 | D = 69 | 1 Nuclear | N | Yes | Yes | Increase in cells |
| mCherry | | | N = 18 | N = 31 | | | | N = 15, | with exclusive |
| | | | PCC 0.29 | PCC 0.43 | | | | D = 80 | nuclear localization |
| | | | | | | | | PCC 0.31 | |

C denotes predominant cytoplasmic expression, D denotes diffuse (both cytoplasmic and nuclear) and N denotes predominant nuclear localization.

### Example 4—Discussion

Ligand-induced translocation of proteins is a versatile tool in biotechnology for applications ranging from understanding tissue-specific conditional expression to adoptive cell therapies. The most well-characterized proteins used for these applications are derived from mammalian proteins and hence the administration of the ligands can cause an off-target response from the activation of endogenous genes. Bacterial-based systems on other hand are both immunogenic and the ligands such as tetracycline and doxycycline can promote antibiotic resistance (Grossman et al., 2016). There is a compelling need for identifying orthogonal systems that are responsive to small-molecule ligands that are well suited for application in mammals.

The inventors aimed to develop an SA-based inducible protein translocation in mammalian cells. SA is the major metabolite of aspirin, and the safety, pharmacokinetics, and pharmacodynamics have been extensively characterized in humans. To identify SA based sensors, the inventors focused on plants since SA is known to be a hormone essential for innate immunity. Although it is well known that the NPR1/3/4 proteins are sensors of SA, the exact affinity and roles of these different proteins in SA sensing is controversial. Arabidopsis thaliana (At) NPR4 is a high-affinity sensor of SA (K_{D}=24 nM) whereas the affinity of NPR1 for SA is (130-200 nM) (Wu et al., 2012; Ding et al., 2018). Comparative studies with both AtNPR1 and Nicotiana tobacco NtNPR1 have shown that the N-terminal domains of these proteins (amino acids 1-315) harbor a strong SA inducible transactivation domain (Han et al., 2019). Both AtNPR1 and NtNPR1 are activated by SA but NtNPR1 accumulates predominantly in the nucleus even in the absence of SA (Maier et al., 2011). AtNPR1 on the other hand is present in the cytoplasm (likely in an oligomeric form) and upon the addition of SA undergoes reduction/conformation change exposing a bipartite nuclear localization signal that facilitates transport to the nucleus. The C-terminal transactivation domain of AtNPR1 (aa 513-593) has been shown to bind directly to SA with a K_{D} of 1.49 μM (Wu et al., 2012). This however is controversial since the putative SA binding domain of NPR1s is predicted to be the conserved⁴²⁹LENRV⁴³³ motif (AtNPR1 numbering) (Maier et al., 2011).

To map the SA mediated translocation domain of AtNPR1, the inventors employed a heterologous expression system using HEK293 cells. This system isolates the SA binding activity of NPR1 and is not subjected to interference from plant defense compounds or signaling cascades. They employed a stable expression system mediated by viral transduction and used mCherry as the live-cell reporter. These results indicate that the AtNPR1 protein, upon the addition of SA, mediates nuclear translocation in HEK293 mammalian cells without the requirement for any accessory plant-derived proteins. At SA concentrations of 1-2.5 mM, the inventors observed that the TAD of NPR1 was sufficient to render the protein in the nucleus upon the addition of the SA. When NPR1 or NPR1-TAD was fused at the C-terminus of mCherry they showed strong nuclear exclusion but translocation mediated by SA was only accomplished with NPR1-TAD. This suggests that the N-terminus of NPR1, if present, must be free to enable SA responsive translocation. By contrast, when NPR1 or NPR1-TAD was fused at the N-terminus of mCherry both constructs showed SA responsive translocation but also basal nuclear expression in the absence of SA.

With respect to the molecular mechanisms of translocation of NPR1 mediated by SA, these results show that the 80 amino acid TAD can mediate nuclear translocation of passenger proteins in the presence of SA and does not require the interaction with the N-terminus of NPR1 to facilitate translocation. As the inventors show, this property is also reversible upon the withdrawal of SA and the proteins revert to their original localization within the cells. It is important to note that unlike the full-length AtNPR1, the AtNPR1-TAD lacks both Cys82 and Cys216 that have been reported to be essential for oligomer monomer transition and subsequent nuclear localization (Table 1). Second, since mCherry-NPR1-TAD is only −36 kDa it is anticipated that passive diffusion through the nuclear pores can enable nuclear translocation. Despite this observation, mCherry-NPR1-TAD shows strong nuclear exclusion in the absence of SA (Table 1). The inventors used the NetNES1.1 server for predicting nuclear export signals within full-length At-NPR1 and the program identified the leucine-rich⁵⁶¹LELGNSSL⁵⁶⁸ as a putative NES within the TAD (la Cour el al., 2004). Broadly, these results are consistent with the aforementioned study that showed that the TAD can directly bind to SA and advances it further by illustrating that they can mediate SA induced translocation. At first glance, these results are not consistent with the known SA binding motif of NPR1 and the recently solved crystal structure of NPR4 bound to SA (Wang et al., 2020). Unlike these results with the TAD, the other studies implicate amino acids 400-500 as the key SA binding regions of NPR1 with Arg432 playing an indispensable role (Maier et al., 2011; Wang et al., 2020; Hermann et al., 2012). A more careful comparison allows us to posit that these results obtained with high concentrations of SA only show that the TAD can mediate nuclear translocation in response to SA but that the true high affinity nanomolar binding region might still be present within amino acids 400-500. As results with full-length NPR1 illustrate, the presence of the full-length protein does not improve SA mediated nuclear translocation of passenger proteins.

From a biotechnological perspective, the NPR1-TAD is attractive as a SA mediated nuclear translocator with the best translocator, mCherry-NPR1-TAD performing nuclear translocation as efficiently as widely utilized the estrogen receptor alpha system (Zhao et al., 2018). There are several advantages to this system. The small size of the TAD minimizes the metabolic load of protein expression. Second, since it is of non-mammalian origin, it is likely immunogenic but again the small size decreases the number of available epitopes. Third, since it is not of microbial origin, it is not likely to be compromised by pre-existing immunity (Stanton et al., 2014; Auslander & Fussenegger, 2016; Gu et al., 2018). From an application standpoint, when fused at the C-terminus of the passenger protein, the basal state is strong nuclear exclusion and hence might be appropriate for DNAse based kill switches in adoptive cell therapy or Cas9 based inducible editors. When fused at the N-terminus of the passenger protein, the induced state is strong nuclear localization and hence this might be attractive for transactivation of gene expression. The inventors recognize, however, that these are conceptual frameworks and this study has only illustrated these behaviors with mCherry. Nonetheless, the availability of small protein domains like the NPR1-TAD that can facilitate SA mediated nuclear translocation in mammalian cells has strong potential for translational applications within living organisms.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. References

The following references, to the extent that they provide exemplary procedural or other details supplementary to those set forth herein, are specifically incorporated herein by reference.

Pokotylo, I., Kravets, V. & Reulland, E. (2019) Salicylic acid binding proteins (SABPs): The hidden forefront of salicylic acid signalling. Int. J. Mol. Sci. 20(18):4377.
Baba, T., T. Ara, M. Hasegawa, Y. Takai, Y. Okumura, M. Baba, K. A. Datsenko, M. Tomita, B. L. Wanner & H. Mori (2006) Construction of Escherichia coli K-12 in-frame, single-gene knockout mutants: the Keio collection. Mol Syst Biol, 2, 2006.0008. /pubmed.ncbi.nlm.nih.gov/16738554/
Nevozhay, D., T. Zal & G. Balázsi (2013) Transferring a synthetic gene circuit from yeast to mammalian cells. Nat Commun. 4, 1451. /pubmed.ncbi.nlm.nih.gov/23385595/
Notka, F., H. J. Linde, A. Dankesreiter, H. H. Niller & N. Lehn (2002) A C-terminal 18 amino acid deletion in MarR in a clinical isolate of Escherichia coli reduces MarR binding properties and increases the MIC of ciprofloxacin. J Antimicrob Chemother, 49, 41-7. /pubmed.ncbi.nlm.nih.gov/11751765/
Seoane, A. S. & S. B. Levy (1995) Characterization of MarR, the repressor of the multiple antibiotic resistance (mar) operon in Escherichia coli. J Bacteriol, 177, 3414-9. /pubmed.ncbi.nlm.nih.gov/7768850/
Sun, P., J. E. Tropea & D. S. Waugh (2011) Enhancing the solubility of recombinant proteins in Escherichia coli by using hexahistidine-tagged maltose-binding protein as a fusion partner. Methods Mol Biol, 705, 259-74. https://pubmed.ncbi.nlm.nih.gov/21125392/.
Feil, R. et al. Ligand-activated site-specific recombination in mice. Proc Natl Acad Sci USA 93, 10887-10890, doi:10.1073/pnas.93.20.10887 (1996).
Zhang, J., Chen, L. & Wang, Y. Drug Inducible CRISPR/Cas Systems. Comput Struct Biotechnol J17, 1171-1177, doi:10.1016/j.csbj.2019.07.015 (2019).
Zhao, C. et al. HIT-Cas9: A CRISPR/Cas9 Genome-Editing Device under Tight and Effective Drug Control. Mol Ther Nucleic Acids 13, 208-219, doi:10.1016/j.omtn.2018.08.022 (2018).
Liu, E. et al. Cord blood NK cells engineered to express IL-15 and a CD19-targeted CAR show long-term persistence and potent antitumor activity. Leukemia 32, 520-531, doi:10.1038/leu.2017.226 (2018).
Fuhrmann-Benzakein, E., Garcia-Gabay, I., Pepper, M. S., Vassalli, J. D. & Herrera, P. L. Inducible and irreversible control of gene expression using a single transgene. Nucleic Acids Res 28, E99, doi:10.1093/nar/28.23.e99 (2000).
Xu, T., Johnson, C. A., Gestwicki, J. E. & Kumar, A. Conditionally controlling nuclear trafficking in yeast by chemical-induced protein dimerization. Nat Prooc 5, 1831-1843, doi:10.1038/nprot.2010.141 (2010).
Di Ventura, B. & Kuhlman, B. Go in! Go out! Inducible control of nuclear localization. Curr Opin Chem Biol 34, 62-71, doi:10.1016/j.cbpa.2016.06.009 (2016).
Malamy, J., Carr, J. P., Klessig, D. F. & Raskin, I. Salicylic Acid: a likely endogenous signal in the resistance response of tobacco to viral infection. Science 250, 1002-1004, doi:10.1126/science.250.4983.1002 (1990).
Metraux, J. P. et al. Increase in salicylic Acid at the onset of systemic acquired resistance in cucumber. Science 250, 1004-1006, doi: 10.1126/science.250.4983.1004 (1990).
Tsuda, K., Sato, M., Stoddard, T., Glazebrook, J. & Katagiri, F. Network properties of robust immunity in plants. PLoS Genet 5, e1000772, doi:10.1371/joumal.pgen.1000772 (2009).
Fu, Z. Q. et al. NPR3 and NPR4 are receptors for the immune signal salicylic acid in plants. Nature 486, 228-232, doi:10.1038/naturel 1162 (2012).
Wu, Y. et al. The Arabidopsis NPR1 protein is a receptor for the plant defense hormone salicylic acid. Cell Rep 1, 639-647, doi:10.1016/j.celrep.2012.05.008 (2012).
Ding, Y. et al. Opposite Roles of Salicylic Acid Receptors NPR1 and NPR3/NPR4 in Transcriptional Regulation of Plant Immunity. Cell 173, 1454-1467 e1415, doi:10.1016/j.cell.2018.03.044 (2018).
Tada, Y. et al. Plant immunity requires conformational changes [corrected] of NPR1 via S-nitrosylation and thioredoxins. Science 321, 952-956, doi:10.1126/science. 1156970 (2008).
Rochon, A., Boyle, P., Wignes, T., Fobert, P. R. & Despres, C. The coactivator function of Arabidopsis NPR1 requires the core of its BTB/POZ domain and the oxidation of C-terminal cysteines. Plant Cell 18, 3670-3685, doi:10.1105/tpc.106.046953 (2006).
Kinkema, M., Fan, W. & Dong, X. Nuclear localization of NPR1 is required for activation of PR gene expression. Plant Cell 12, 2339-2350, doi: 10.1105/tpc.12.12.2339 (2000).
Chen, X., Zaro, J. L. & Shen, W. C. Fusion protein linkers: property, design and functionality. Adv Drug Deliv Rev 65, 1357-1369, doi:10.1016/j.addr.2012.09.039 (2013).
Grossman, T. H. Tetracycline Antibiotics and Resistance. Cold Spring Harb Perspect Med 6, a025387, doi:10.1101/cshperspect.a025387 (2016).
Han, G. Z. Origin and evolution of the plant immune system. New Phytol 222, 70-83, doi:10.1111/nph.15596 (2019).
Maier, F. et al. NONEXPRESSOR OF PATHOGENESIS-RELATED PROTEINS1 (NPR1) and some NPR1-related proteins are sensitive to salicylic acid. Mol Plant Pathol 12, 73-91, doi:10.1111/j.1364-3703.2010.00653.x (2011).
Wang, W. et al. Structural basis of salicylic acid perception by Arabidopsis NPR proteins. Nature 586, 311-316, doi:10.1038/s41586-020-2596-y (2020).
Hermann, M. et al. The Arabidopsis NIMIN proteins affect NPR1 differentially. Front Plant Sci 4, 88, doi:10.3389/fpls.2013.00088 (2013).
Stanton, B. C. et al. Systematic transfer of prokaryotic sensors and circuits to mammalian cells. ACS Synth Biol 3, 880-891, doi:10.1021/sb5002856 (2014).
Auslander, S. & Fussenegger, M. Engineering Gene Circuits for Mammalian Cell-Based Applications. Cold Spring Harb Perspect Biol 8, doi:10.1101/cshperspect.a023895 (2016).
Gu, X., He, D., Li, C., Wang, H. & Yang, G. Development of Inducible CD19-CAR T Cells with a Tet-On System for Controlled Activity and Enhanced Clinical Safety. Int J Mol Sci 19, doi:10.3390/ijms19113455 (2018).
Kelley, L., Mezulis, S., Yates, C. el al. The Phyre2 web portal for protein modeling, prediction and analysis. Nat Protoc 10, 845-858, doi.10.1038/nprot.2015.053 (2015). Reynolds, C. R., Islam, S. A., & Stemnberg M. J. E., “EzMol: A Web Server Wizard for the Rapid Visualization and Image Production of Protein and Nucleic Acid Structures”, J Mol Biol, 430, 2244-2248, doi: 10.1016/j.jmb.2018.01.013 (2018).
la Cour T, Kiemer L. Mølgaard A, Gupta R, Skriver K, Brunak S, “Analysis and prediction of leucine-rich nuclear export signals.” Protein Eng Des Sel.; 17(6), 527-536. doi:10.1093/protein/gzh062 (2004).

1. A method of providing modulated gene expression in a cell comprising:
(a) providing an engineered cell comprising
(i) a target gene under the control of a transcription element modulated by a transcription modulating protein; and
(ii) a chimeric molecule comprising the transcription modulating protein or functional domain thereof, a salicylic acid binding domain and optionally a further nuclear localization signal, and
(b) contacting said cell with salicylic acid,
thereby modulating expression of said target gene.

2. The method of claim 1, wherein said chimeric molecule is encoded from an extrachromosomal element in said cell.

3. The method of claim 2, wherein said method further comprises transferring said extrachromosomal element into said cell, such as by liposome or nanoparticle delivery.

4. The method of claim 1, wherein said chimeric molecule is expressed from a chromosomal element in said cell.

5. The method of claim 1, wherein said transcription modulating protein or functional domain therefore is herpesvirus herpes simplex VP16, FoxA or MyoD.

6. The method of claim 1, wherein the transcription modulating protein or functional domain thereof comprises (i) CRISPR associated protein 9 (Cas9), a dead Cas9 (dCas9), or Cpf1 and said cell is engineered to constitutively express an sgRNA with specificity for the transcription element or (ii) a transcription activator-like effector (TALE).

7. The method of claim 1, wherein the salicylic acid binding domain is from a different protein than the nuclear localization signal.

8. The method of claim 1, wherein the salicylic acid binding domain is from the same protein as the nuclear localization signal.

9. The method of claim 1, wherein the salicylic acid binding domain is from non-expressor of pathogenesis related gene (NPR) to a TGA transcription factor.

10. The method of claim 1, wherein the nuclear localization signal is from SV40.

11. The method of claim 1, wherein said cell is a mammalian cell, such as one located in a living subject.

12. The method of claim 1, wherein said target gene and said transcription element are native to said cell.

13. The method of claim 1, wherein said target gene and said transcription element are not native to said cell.

14. The method of claim 1, wherein said transcription modulating protein or functional domain thereof is a negative modulator of transcription.

15. The method of claim 1, wherein said transcription modulating protein or functional domain thereof is an inducer of transcription.

16. The method of claim 1, wherein the target gene is a chimeric antigen receptor, an antibody, a toxin, a cytokine, an enzyme, a hormone, or a receptor ligand.

17. The method of claim 1, wherein the target gene is insulin, a type I interferon, a type II interferon, a type III interferon, an interleukin, erythropoietin, or tissue plasminogen activator.

18. The method of claim 1, wherein the transcription modulating protein or functional domain thereof comprises a solubility/folding domain.

19. A method of inducing apoptosis in a mammalian cell (e.g., an immune cell), such as one located in a living subject, comprising:
(a) providing a mammalian cell engineered to contain a cytotoxic gene (e.g., a DNase) fused to a salicylic acid binding domain and nuclear localization signal; and
(b) contacting said cell with salicylic acid,
thereby translocating the cytotoxic gene product into the nucleus, resulting in apoptosis.

20. A method of providing modulated gene expression in a cell, such as one located in in living organism, comprising:
(a) providing a cell comprising:
(i) a heterologous target gene under the control of a transcription element modulated by a salicylic acid responsive transcription modulating protein; and
(ii) a chimeric molecule comprising a functional domain of the salicylic acid responsive transcription modulating protein, a salicylic acid binding domain, and nuclear localization signal, and
(b) contacting said cell with salicylic acid,
thereby modulating expression of said target gene.

21. The method of claim 20, wherein said cell is a mammalian cell or a plant cell.

22. The method of claim 20, wherein said salicylic acid responsive transcription modulating protein further comprises a nuclear localization domain and/or a solubility/folding domain and/or a transactivation domain.

23. The method of claim 22, wherein the nuclear localization domain is from SV40 and/or the transactivation domain comprises herpes simplex virus VP16, FoxA or MyoD.

24. The method of claim 21, wherein the salicylic acid responsive transcription modulating protein is from a plant cell.

25. The method of claim 24, wherein the salicylic acid responsive transcription modulating protein is non-expressor of pathogenesis related gene (NPR) or TGA transcription factor.

26. The method of claim 21, wherein the target gene is not native to said cell.

27. The method of claim 21, wherein the salicylic acid responsive transcription modulating protein and transcription element are not native to said cell.

28. The method of claim 21, wherein the salicylic acid responsive transcription modulating protein, transcription element and target gene are not native to said cell.

29. The method of claim 21, wherein the target gene is a chimeric antigen receptor, an antibody, a toxin, a cytokine, an enzyme, a hormone, or a receptor ligand.

30. The method of claim 21, wherein the target gene is insulin, a type I interferon, a type II interferon, a type III interferon, an interleukin, erythropoietin, or tissue plasminogen activator.

31. The method of claim 21, wherein said cell is a eukaryotic cell.

32. The method of claim 21, wherein said salicylic acid responsive transcription modulating protein is a repressor of transcription.

33. The method of claim 21, wherein said salicylic acid responsive transcription modulating protein is an inducer of transcription.

34. A method of providing modulated gene expression in a cell comprising:
(a) providing a host cell comprising:
(i) a heterologous target gene under the control of a transcription element modulated by a salicylic acid responsive transcription factor; and
(ii) a salicylic acid responsive transcription factor, and
(b) contacting said cell with salicylic acid,
thereby modulating expression of said target gene.

35. A method of inducible gene editing in a cell comprising:
(a) providing an engineered cell comprising:
(i) a target gene that needs to be edited; and
(ii) a chimeric molecule comprising a nuclease or functional domain thereof, a salicylic acid binding domain and nuclear localization signal, and
(b) contacting said cell with salicylic acid,
thereby editing the target gene.
